(19) United States
(12) Patent Application Publication
(10) Pub. No.: US 2018/0185495 A1
(43) Pub. Date: Jul. 5, 2018

(54) THERAPEUTIC COMPOSITIONS OF ALPHA-L-IDURONIDASE, IDURONATE-2-SULFATASE, AND ALPHA-GALACTOSIDASE A AND METHODS OF USE THEREOF

(71) Applicant: Alexion Pharmaceuticals, Inc., New Haven, CT (US)

(72) Inventors: Jonathan Heller, Belmont, MA (US); Mohammed Qatanani, Needham, MA (US); Gregory Grabowski, Lexington, MA (US)

(73) Assignee: Alexion Pharmaceuticals, Inc., New Haven, CT (US)

(21) Appl. No.: 15/525,690

(22) PCT Filed: Nov. 10, 2015
(86) PCT No.: PCT/US2015/059966

§ 371 Date: May 10, 2017

Related U.S. Application Data
(60) Provisional application No. 62/173,091, filed on Jun. 9, 2015.
(60) Provisional application No. 62/093,316, filed on Dec. 17, 2014.
(60) Provisional application No. 62/080,838, filed on Nov. 17, 2014.
(60) Provisional application No. 62/077,654, filed on Nov. 10, 2014.

(51) Int. Cl. A61K 47/42; A61K 38/47; A61K 9/00; A61K 38/46; C12N 9/24; C12N 9/16; C12N 9/40

(52) U.S. Cl. CPC A61K 47/42; A61K 38/47; C12Y 302/01076; A61K 9/0019; C12N 9/2465; C12Y 301/06013; C12Y 302/01022; C12N 9/2402; C12N 9/16; A61K 38/465

(57) ABSTRACT

The present invention provides pharmaceutical compositions comprising an a blood brain barrier peptide and a human peptide, such as an alpha-L-iduronidase (IDUA) protein, an iduronate-2-sulfatase protein (IDS) protein, or an a galactosidase A protein (α-Gal A) protein. The invention further provides methods of use for treating Mucopolysaccharidosis type I (MPS I), including Hurler Syndrome, Hurler-Scheie Syndrome and Scheie Syndrome; methods of use for treating Hunter syndrome; and methods of use for treating Fabry disease.

### RELATED APPLICATIONS

This application claims priority to U.S. Provisional Application No. 62/077,654, filed on Nov. 10, 2014; U.S. Provisional Application No. 62/080,838, filed on Nov. 17, 2014; U.S. Provisional Application No. 62/093,316, filed on Dec. 17, 2014; and U.S. Provisional Application No. 62/173,091, filed on Jun. 9, 2015. The entire contents of each of the foregoing applications are expressly incorporated herein by reference.

### BACKGROUND

Mucopolysaccharidosis type I (MPS I) is a genetic disorder that affects numerous body systems and leads to organ damage. MPS I is caused by defects in the alpha-L-iduronidase (IUDA) gene. MPS IH is known as Hurler Syndrome, and MPS IS is known as Scheie syndrome, which has an attenuated phenotype compared to Hurler Syndrome. Signs of MPS I may include stiffened joints, skeletal abnormalities, carpal tunnel syndrome, cardiac (valvular) disease, recurrent upper airway infections, obstructive airway disease (sleep apnea), corneal clouding, spinal cord compression, hepatosplenomegaly/splenomegaly, inguinal or umbilical hernia, hearing loss, mental retardation, coarse facial features, communicating hydrocephalus, and abnormally shaped teeth.

Alpha-L-iduronidase (IDUA; EC 3.2.1.76) is an enzyme which catalysis the hydrolysis of unsulfated alpha-L-iduronosidic linkages in dermatan and heparan sulfates, and is involved in the degradation of the glycosaminoglycans dermatan sulfate and heparan sulfate. In MPS I, IDUA deficiency results in the lysosomal accumulation of heparan sulfate and dermatan sulfate, which causes a variety of complications in the respiratory, cardiac, and brain and nervous systems, including, but not limited to, cognitive abnormalities, hydrocephalus, hypertrophic cervical pachymeningitis, nerve compression, and/or behavioral abnormalities.

Since MPS I is a multisystemic disease, treatment of MPS I patients is complex and involves the treatment of its many signs and symptoms. To date, no cure is available for MPS I. Enzyme replacement therapy using intravenous IDUA has been performed, however, IDUA has been shown not to cross the blood brain barrier. Thus, current IDUA enzyme replacement therapies do not help solve the neurological involvement experienced by MPS I patients.

Hunter syndrome (mucopolysaccharidosis type II, MPS-II), characterized by variable phenotypes from severe mental retardation, skeletal deformities, and stiff joints to a relatively mild course, is caused by the deficiency in the activity of IDS, leading to the lysosomal accumulation of heparin sulfate and dermatan sulfate fragments and their exertion in urine (Neufeld et al., The Metabolic Basis of Inherited Disease, eds. Scriver et al., pp. 1565-1587, McGraw-Hill, New York 1989). This clinical heterogeneity has been suggested to reflect different mutations of IDS, affecting IDS's expression, stability or function. Hunter syndrome is the only mucopolysaccharidosis that is X chromosome-linked (Neufeld et al., The Metabolic Basis of Inherited Disease, eds. Scriver et al., pp. 1565-1587, McGraw-Hill, New York 1989).

IDS is an exosulfatase in lysosomes whose function involves hydrolyzing the C2-sulfate ester bond from nonreducing-terminal iduronic acid residues in the glycosaminoglycans heparin sulfate and dermatan sulfate (Neufeld et al., The Metabolic Basis of Inherited Disease, eds. Scriver et al., pp. 1565-1587, McGraw-Hill, New York 1989). IDS belongs to a family of at least nine sulfatases that hydrolyze sulfate esters in human cells. These lysosomal enzymes acts on sulfated monosaccharide residues in various substrates except microsomal steroid sulfatase, also known as arylsulfatase C, which acts on sulfated 3beta-hydroxysteroids (Neufeld et al., The Metabolic Basis of Inherited Disease, eds. Scriver et al., pp. 1565-1587, McGraw-Hill, New York 1989; Shapiro, The Metabolic Basis of Inherited Disease, eds. Scriver et al., pp. 1945-1964, McGraw-Hill, New York 1989).

Since Hunter syndrome is a multisystemic disease, treatment of Hunter syndrome patients is complex and involves the treatment of its many signs and symptoms. To date, no cure is available for Hunter syndrome. Enzyme replacement therapy using intravenous IDS has been performed, however, IDS has been shown not to cross the blood brain barrier. Thus, current IDS enzyme replacement therapies do not help solve the neurological involvement experienced by Hunter syndrome patients.

Fabry disease is an X-linked inborn error of glycosphingolipid metabolism caused by deficient lysosomal α-galactosidase A (α-Gal A) activity (Desnick et al., The Metabolic and Molecular Bases of Inherited Disease, 8^{th} Edition, Scriver et al. ed., pp. 3733-3774, McGraw-Hill, New York 2001; Brady et al., N. Engl. J. Med. 1967; 276, 1163-1167). The α-Gal A gene has been mapped to Xq22, (Bishop et al., Am. J. Hum. Genet. 1985; 37: A144), and the full-length cDNA and entire 12-kb genomic sequences encoding α-Gal A have been reported (Calhoun et al., Proc. Natl. Acad. Sci. USA 1985; 82: 7364-7368; Bishop et al., Proc. Natl. Acad. Sci USA 1986; 83 4859-4863; Tsuji et al., Eur, J, Biochem, 1987; 2750280; and Kornreich et al., Nucleic Acids Res. 1989; 17: 3301-3302). There is a marked genetic heterogeneity of mutations that cause Fabry disease (The Metabolic and Molecular Bases of Inherited Disease, 8th Edition 2001, Scriver et al., ed., pp. 3733-3774, McGraw-Hill, New York.; Eng et al., Am. J. Hum. Genet. 1993; 53: 1186-1197; Eng et al., Mol. Med. 1997; 3: 174-182; and Davies et al., Eur. J. Hum. Genet. 1996; 4: 219-224). To date, a variety of missense, nonsense, and splicing mutations, in addition to small deletions and insertions, and larger gene rearrangements have been reported. The enzymatic defects associated with these mutations lead to the progressive deposition of neutral glycosphingolipids with α-galactosyl residues, predominantly globotriaosylceramide (GL-3), in body fluids and tissue lysosomes.

The frequency of the disease is estimated to be about 1:40,000 in males, and is reported throughout the world within different ethnic groups. In classically affected males, the clinical manifestations include angiokeratoma, acroparesthesias, hypohidrosis, and characteristic corneal and lenticular opacities (The Metabolic and Molecular Bases of Inherited Disease, 8^{th} Edition, 2001, Scriver et al., ed., pp. 3733-3774, McGraw-Hill, New York). The affected male's life expectancy is reduced, and death usually occurs in the fourth or fifth decade as a result of vascular disease of the heart, brain, and/or kidneys. In contrast, patients with the milder “cardiac variant” normally have 5-15% of normal α-Gal A activity, and present with left ventricular hypertrophy or a cardiomyopathy. These cardiac variant patients remain essentially asymptomatic when their classically affected counterparts are severely compromised. Recently, cardiac variants were found in 11% of adult male patients with unexplained left ventricular hypertrophic cardiomyopathy, suggesting that Fabry disease may be more frequent than previously estimated (Nakao et al., N. Engl. J. Med. 1995; 333: 288-293).

Fabry disease also manifests in both the peripheral nervous system and the central nervous system (CNS), with globotriaosylceramide accumulation in Schwann cells and dorsal root ganglia, as well as in neurons of the CNS. Cerebrovasculopathy secondary to Fabry disease results in an increased incidence of stroke in affected subjects. See, e.g., Schiffmann and Moore, Neurological manifestations of Fabry disease. In: Mehta A, Beck M, Sunder-Plassmann G, editors. Fabry Disease: Perspectives from 5 Years of FOS. (Oxford: Oxford PharmaGenesis; 2006), Chapter 22.

Since Fabry disease is a multisystemic disease, treatment of Fabry disease patients is complex and involves the treatment of its many signs and symptoms. To date, no cure is available for Fabry disease. Enzyme replacement therapy using α-Gal A has been performed, however, α-Gal A has been shown not to cross the blood brain barrier. Thus, current α-Gal A enzyme replacement therapies do not help solve the neurological involvement experienced by Fabry disease patients.

### SUMMARY

Although enzyme replacement therapy using intravenous IDUA, IDS and a galactosidase A has been performed, IDUA, IDS and a galactosidase A have been shown not to cross the blood brain barrier, and are thus not effective treatments of central nervous system involvement by MPS I, Hunter syndrome and Fabry diseases. The methods and compositions described herein address three factors that are important in delivering a therapeutically significant level of IDUA, IDS or a galactosidase A protein across the blood brain barrier in order to treat MPS I, Hunter syndrome or Fabry diseases: 1) construction of an IDUA protein or complex, an IDS protein or complex, or an α galactosidase A protein or complex that can cross the blood brain barrier; 2) determination of the proper therapeutic amount of the IDUA protein or complex, the IDS protein or complex, or the α galactosidase A protein or complex (e.g., relative amounts of the components in a complex); and 3) retention of IDUA, IDS, or α galactosidase A enzymatic activity once across the blood brain barrier with sufficient activity to decrease the amounts of offending substrates, i.e., retention of IDUA, IDS or α galactosidase A enzymatic activity in a therapeutically effective amount. The instant invention addresses these factors for the first time, by providing a composition comprising a blood-brain carrier peptide (“BBB carrier peptide) and either an enzymatically active iduronate-2-sulfatase (IDS) protein, IDUA protein, or α galactosidase A (α-Gal A) protein. The instant invention also establishes therapeutically effective doses of the IDUA:BBB carrier peptide composition, the IDS:BBB carrier peptide composition, and the α-Gal A:BBB carrier peptide composition.

Thus, in a first aspect, the invention provides compositions comprising a human protein (e.g., IDUA, IDS, or α-Gal A) and a carrier peptide that facilitates the transport of the human protein across the blood-brain barrier (BBB), resulting in delivery of the human protein into the central nervous system of subjects and, thereby, treating the neurological deficits associated with MPS I, including Hurler Syndrome and Scheie Syndrome; Hunter syndrome; or Fabry disease.

In some embodiments, the BBB carrier peptide comprises a first portion comprising a transferrin-receptor binding site of a transferrin, or a receptor binding domain of an apolipoprotein, linked to a second portion comprising a hydrophilic segment of from 4-50 hydrophilic amino acids. In some embodiments, the first portion comprises a receptor-binding domain of an apolipoprotein, selected from the receptor-binding domain of ApoA, ApoB, ApoC, ApoD, ApoE, ApoE2, ApoE3, and ApoE4. In some embodiments, the hydrophilic amino acids are selected from the group consisting of arginine, asparagine, aspartic acid, glutamic acid, glutamine, histidine, lysine, serine, threonine, and tyrosine. In some embodiments, the BBB carrier peptide comprises or consists of the sequence K-K-K-K-K-K-K-K-K-K-K-K-K-K-K-K-L-R-V-R-L-A-S-H-L-R-K-L-R-K-R-L-L-R-D-A (SEQ ID NO:45). In some embodiments, the IDUA, IDS or α galactosidase A proteins are non-covalently complexed with the blood-brain barrier carrier peptide.

In some embodiments, the IDUA protein comprises SEQ ID NO:53. In other embodiments, the IDUA protein is at least 80% identical to SEQ ID NO:53. In another embodiment, the IDUA protein is at least 85% identical to SEQ ID NO:53. In another embodiment, the IDUA protein is at least 90% identical to SEQ ID NO:53. In another embodiment, the IDUA protein is at least 95% identical to SEQ ID NO:53. In another embodiment, the IDUA protein is at least 80, 81, 82, 83, 84, 85, 86, 87, 88, 89, 90, 91, 92, 93, 94, 95, 96, 97, 98 or 99% identical to SEQ ID NO:53. In other embodiments, the IDUA protein comprises amino acids 20-653 of SEQ ID NO:53. Other IDUA proteins are well known in the art and are described in, for example, U.S. Pat. No. 6,426,208, the entire contents of which are expressly incorporated herein by reference.

In some embodiments, the IDS protein comprises SEQ ID NO:55. In other embodiments, the IDUA protein is at least 80% identical to SEQ ID NO:55. In another embodiment, the IDUA protein is at least 85% identical to SEQ ID NO:55. In another embodiment, the IDUA protein is at least 90% identical to SEQ ID NO:55. In another embodiment, the IDUA protein is at least 95% identical to SEQ ID NO:55. In another embodiment, the IDUA protein is at least 80, 81, 82, 83, 84, 85, 86, 87, 88, 89, 90, 91, 92, 93, 94, 95, 96, 97, 98 or 99% identical to SEQ ID NO:55. In other embodiments, the IDUA protein comprises amino acids 26-550 of SEQ ID NO:55.

In some embodiments, the α-galactosidase A protein comprises SEQ ID NO:56. In other embodiments, the IDUA protein is at least 80% identical to SEQ ID NO:56. In another embodiment, the IDUA protein is at least 85% identical to SEQ ID NO:56. In another embodiment, the IDUA protein is at least 90% identical to SEQ ID NO:56. In another embodiment, the IDUA protein is at least 95% identical to SEQ ID NO:56. In another embodiment, the IDUA protein is at least 80, 81, 82, 83, 84, 85, 86, 87, 88, 89, 90, 91, 92, 93, 94, 95, 96, 97, 98 or 99% identical to SEQ ID NO:56. In other embodiments, the IDUA protein comprises amino acids 32-429 of SEQ ID NO:56.

In some embodiments, the human protein (e.g., the IDUA protein, the IDS protein, or the α-galactosidase A protein) and BBB carrier peptide are present in a molar ratio of at least 1:2, e.g., at least 1:2.5, at least 1:3, at least 1:4, at least 1:5, at least 1:6, at least 1:7, at least 1:8, at least 1:9, at least 1:10, at least 1:11, at least 1:12, at least 1:13, at least 1:14, at least 1:15, at least 1:20, or higher. In one embodiment, the human protein and the BBB carrier peptide are present in a molar ratio from about 1:10 to about 1:170; a molar ratio from about 1:50 to about 1:170; a molar ratio from about 1:100 to about 1:170; a molar ratio from about 1:160 to about 1:170; a molar ratio from about 1:165 to about 1:170.

In one embodiment, the human protein (e.g., the IDUA protein, the IDS protein, or the α-galactosidase A protein) and the BBB carrier peptide are present in a molar ratio of about 1:10; about 1:25; about 1:50; about 1:75; about 1:100; about 1:125; about 1:130; about 1:140; about 1:150; about 1:160; about 1:165; about 1:167; or about 1:170.

In further embodiments, the IDUA protein and BBB carrier peptide are present in any of the foregoing molar ratios, wherein the human protein (e.g., the IDUA protein, the IDS protein, or the α-galactosidase A protein) is formulated for administration at a dose of about 0.2-50 mg of human protein/kg of body weight, e.g., 0.2 mg/kg, 0.3 mg/kg, 0.4 mg/kg, 0.5 mg/kg, 0.58 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, or 50 mg/kg. In another embodiment, the human protein is formulated for administration at a dose of about 5 mg/kg to about 15 mg/kg; about 5 mg/kg to about 25 mg/kg; about 25 mg/kg to about 50 mg/kg; or about 50 mg/kg.

In some embodiments, the human protein (e.g., the IDUA protein, the IDS protein, or the α-galactosidase A protein) is administered to a subject in a dose of about 1 mg to about 65 mg; about 5 mg to about 60 mg; about 10 mg to about 55 mg; about 20 mg to about 45 mg; or about 30 mg. In one embodiment, the pharmaceutical composition of the invention comprises about 1, 5, 10, 15, 20, 25, 30, 35, 36, 37, 38, 39, 40, 41, 42, 43, 44, 45, 46, 47, 48, 49, 50, 51, 52, 53, 54, 55, 56, 57, 58, 59 or 60 mg of the human protein (e.g., the IDUA protein, the IDS protein, or the α-galactosidase A protein).

In some embodiments, the human protein (e.g., the IDUA protein, IDS protein, or α-galactosidase A protein) is administered in a dose of at least about 10 nmole to about 750 nmole; about 10 nmole to about 350 nmole; about 10 nmole to about 600 nmole; about 100 nmole to about 600 nmole; about 300 nmole to about 500 nmole; about 350 nmole to about 450 nmole; or about 10, 25, 50, 75, 100, 125, 150, 175, 200, 225, 250,275, 300, 325, 350, 375, 400, 425, 450, 475, 500, 525, 550, 575, 600, 625, 650, 675, 700, 725, or 750 nmole.

In some embodiments, the BBB carrier peptide is administered in a dose of about 1 mg/kg to about 10 mg/kg. In one embodiment, the BBB carrier peptide is administered in a dose of about 2 mg/kg to about 8 mg/kg. In one embodiment, the BBB carrier peptide is administered in a dose of about 5 mg/kg to about 8 mg/kg. In one embodiment, the BBB carrier peptide is administered in a dose of about 6 mg/kg to about 7 mg/kg. In one embodiment, the BBB carrier peptide is administered in a dose of about 10 mg/kg; about 9 mg/kg; about 8 mg/kg; about 7 mg/kg; about 6.5 mg/kg; about 6 mg/kg; about 5 mg/kg; about 4 mg/kg; about 3 mg/kg; about 2 mg/kg; or about 1 mg/kg.

In some embodiments, the BBB carrier peptide is administered to a subject in a dose of about 3 μmole to about 150 μmole. In another embodiment, the BBB carrier peptide is administered in a dose of about 10 μmole to about 100 μmole. In another embodiment, the BBB carrier peptide is administered in a dose of about 25 μmole to about 75 μmole. In one embodiment, the BBB carrier peptide is administered in a dose of about 50 mole. In another embodiment, the BBB carrier peptide is administered in a dose of about 3, 5, 10, 15, 20, 25, 50, 75, 80, 85, 90, 95, 100, 105, 110, 115, 120, 125, or 130 μmole.

In one embodiment, the BBB carrier peptide is administered to a subject in a dose of about 10 mg to about 600 mg; about 75 mg to about 500 mg; about 375 mg to about 600 mg; about 75 mg to about 375 mg; about 75 mg to about 600 mg; or about 487.5 mg. In another embodiment, the BBB carrier peptide is administered to a subject in a dose of about 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, or 600 mg of the BBB carrier peptide.

In one embodiment, the human protein (e.g., the IDUA protein, the IDS protein, or the α-galactosidase A protein) and the BBB carrier peptide are administered in a molar ratio from about 1:10 to about 1:170; about 1:50 to about 1:170; about 1:100 to about 1:170; about 1:160 to about 1:170; about 1:165 to about 1:170; about 1:10; about 1:25; about 1:50; about 1:75; about 1:100; about 1:125; about 1:130; about 1:140; about 1:150; about 1:160; about 1:165; about 1:167; or about 1:170.

In a particular embodiment, the human protein (e.g., the IDUA protein, the IDS protein, or the α-galactosidase A protein) and the BBB carrier peptide are present in a molar ratio of about 1:155 to about 1:175 (e.g., about 1:160, 1:165, 1:167, 1:170), wherein the human protein is formulated for administration at a dose of about 0.2-5 mg of human protein/kg of body weight, e.g., about 0.2 mg/kg, 0.3 mg/kg, 0.4 mg/kg, 0.5 mg/kg, about 0.58 mg/kg, about 1 mg/kg, about 2 mg/kg, about 3 mg/kg, about 4 mg/kg or about 5 mg/kg. In one embodiment, the human protein (e.g., the IDUA protein, the IDS protein, or the α-galactosidase A protein) and the BBB carrier peptide are present in a molar ratio of about 1:155 to about 1:175 (e.g., about 1:160, 1:165, 1:167, or 1:170), wherein the human protein is formulated for administration in an amount effective to reduce and/or arrest further accumulation of heparan sulfate levels in visceral tissue or urine of a subject.

In some embodiments, the human protein (e.g., the IDUA protein, the IDS protein, or the α-galactosidase A protein) and BBB carrier peptide are administered in any of the foregoing molar ratios, wherein the human protein is administered at a dose of about 0.2-5, 0.5-5, 0.5-4, 0.5-3, 0.5-2, or 0.5-1 mg/kg. In a particular embodiment, the human protein and the BBB carrier peptide are administered in a molar ratio of about 1:155 to about 1:175 (e.g., about 1:160, 1:164, 1:165, 1:166, 1:167, 1:168, 1:169, or 1:170), wherein the human protein is administered at a dose of about 0.2-5 mg of human protein/kg of body weight, e.g., about 0.2 mg/kg, 0.3 mg/kg, 0.4 mg/kg, 0.5 mg/kg, about 0.58 mg/kg, about 1 mg/kg, about 2 mg/kg, about 3 mg/kg, about 4 mg/kg or about 5 mg/kg. In other embodiments, the human protein (e.g., the IDUA protein, the IDS protein, or the α-galactosidase A protein) and the BBB carrier peptide are administered in a molar ratio of about 1:155 to about 1:175 (e.g., about 1:160, 1:165, 1:167, or 1:170), wherein the human protein is administered in an amount effective to reduce, and/or arrest further accumulation of heparan sulfate levels in visceral tissue or urine of a subject.

In one embodiment, the human protein (e.g., the IDUA protein, the IDS protein, or the α-galactosidase A protein) and the BBB carrier peptide are administered in a mg/kg dose ratio of 1:0.5 to about 1:15; 1:2 to about 1:13; 1:5 to about 1:9; 1:8 to about 1:11; or about 1:15, 1:14, 1:13, 1:12, 1:11, 1:10, 1:9, 1:8, 1:7, 1:6, 1:5, 1:4, 1:3, 1:2, 1:1, or 1:0.5.

In some embodiments, the human protein (e.g., the IDUA protein, the IDS protein, or the α-galactosidase A protein) and BBB carrier peptide are administered in any of the foregoing mg/kg dose ratios, wherein the human protein is administered at a dose of about 0.2-5, 0.5-5, 0.5-4, 0.5-3, 0.5-2, or 0.5-1 mg/kg. In a particular embodiment, the human protein and the BBB carrier peptide are administered in a mg/kg dose ratio of about 1:6 to about 1:12 (e.g., about 1:7, 1:8, 1:9, 1:10, or 1:11), wherein the human protein is administered at a dose of about 0.2 to about 5 mg of IDUA/kg of body weight, e.g., about 0.2 mg/kg, 0.3 mg/kg, 0.4 mg/kg, 0.5 mg/kg, about 0.58 mg/kg, about 1 mg/kg, about 2 mg/kg, about 3 mg/kg, about 4 mg/kg or about 5 mg/kg. In some embodiments, the human protein and the BBB carrier peptide are administered in a mg/kg dose ratio of about 1:6 to about 1:12 (e.g., about 1:7, 1:8, 1:9, 1:10, or 1:11), wherein the human protein is administered in an amount effective to reduce, and/or arrest further accumulation of accumulation of heparan sulfate levels in visceral tissue or urine of a subject.

In one embodiment, the molar ratio of the human protein (e.g., the IDUA protein, the IDS protein, or the α-galactosidase A protein) to the blood-brain barrier carrier peptide is 1:167. In one embodiment, the human protein (e.g., the IDUA protein, the IDS protein, or the α-galactosidase A protein) and the BBB carrier peptide are administered in a mg/kg dose ratio of 1:11, wherein the human protein is administered in an amount effective to reduce and/or arrest further accumulation of the level of heparan sulfate in a subject. In one embodiment, the human protein (e.g., the IDUA protein, the IDS protein, or the α-galactosidase A protein) is administered at a dose of about 0.58 mg/kg, and the BBB carrier peptide is administered at a dose of about 6.5 mg/kg. In another embodiment, the human protein (e.g., the IDUA protein, the IDS protein, or the α-galactosidase A protein) is administered at a dose of about 0.21 nmole and the BBB carrier peptide is administered at a dose of about 35 nmole. In another embodiment, the human protein (e.g., the IDUA protein, the IDS protein, or the α-galactosidase A protein) is non-covalently complexed with the blood-brain carrier peptide.

In one embodiment, the pharmaceutical compositions of the invention are administered once weekly. In another embodiment, the pharmaceutical compositions of the invention are administered twice weekly.

In a further aspect, provided are methods for delivering an enzymatically active IDUA protein, an enzymatically active IDS protein, or an enzymatically active α-galactosidase A protein to the central nervous system of a subject, the methods comprising administering a pharmaceutical composition as described herein to the subject. In another aspect, the invention provides a method of delivering an enzymatically active IDUA protein to the heart of a subject having MPS I, the method comprising administering a pharmaceutical composition of the invention to the subject, thereby delivering the IDUA protein to the heart of the subject having MPS I.

The invention also provides a method of delivering an enzymatically active IDS protein to the heart of a subject having Hunter syndrome, the method comprising administering a pharmaceutical composition of the invention to the subject, thereby delivering the IDS protein to the heart of the subject having Hunter syndrome.

The invention also provides a method of delivering an enzymatically active α-galactosidase A protein to the heart of a subject having Fabry disease, the method comprising administering a pharmaceutical composition of the invention to the subject, thereby delivering the α-galactosidase A protein to the heart of the subject having Fabry disease.

In another aspect, the invention provides a method of delivering an enzymatically active IDUA protein to the kidneys of a subject having MPS I, the method comprising administering a pharmaceutical composition of the invention to the subject, thereby delivering the IDUA protein to the kidneys of the subject having MPS I.

The invention also provides a method of delivering an enzymatically active IDS protein to the kidneys of a subject having Hunter syndrome, the method comprising administering a pharmaceutical composition of the invention to the subject, thereby delivering the IDS protein to the kidneys of the subject having Hunter syndrome.

The invention also provides a method of delivering an enzymatically active α-galactosidase A protein to the kidneys of a subject having Fabry disease, the method comprising administering a pharmaceutical composition of the invention to the subject, thereby delivering the α-galactosidase A protein to the kidneys of the subject having Fabry disease.

In an additional aspect, provided are methods for treating a subject having MPS I, Hunter syndrome, or Fabry disease, the methods comprising administering to the subject a therapeutically effective amount of a pharmaceutical composition as described herein, i.e., compositions comprising a therapeutically effective amount of recombinant human IDUA and a BBB carrier peptide, recombinant human IDS and a BBB carrier peptide, or recombinant human α-galactosidase A and a BBB carrier peptide.

In another aspect, the invention provides a method of treating a subject having MPS I or Hunter syndrome, the method comprising administering to the subject a therapeutically effective amount of a pharmaceutical composition of the invention wherein the level of heparan sulfate in the subject is reduced and/or further accumulation of heparan sulfate is arrested in the subject, thereby treating the subject having MPS I or Hunter syndrome.

In another aspect, the invention provides a method of increasing hydrolysis of unsulfated alpha-L-iduronosidic linkages in dermatan sulfate and heparan sulfate in the brain of a subject having MPS I, the method comprising administering to the subject a therapeutically effective amount of a pharmaceutical composition of the invention, thereby increasing the hydrolysis of unsulfated alpha-L-iduronosidic linkages in dermatan sulfate and heapran sulfate in the brain of the subject having MPS I.

In another aspect, the invention provides a method of increasing hydrolysis of C2-sulfate ester bonds from nonreducing-terminal iduronic residues in the heparin sulfate and dermatan sulfate in the brain of a subject having Hunter syndrome, the method comprising administering to the subject a therapeutically effective amount of a pharmaceutical composition of the invention, thereby increasing the hydrolysis of C2-sulfate ester bonds from nonreducing-terminal iduronic residues in the heparin sulfate and dermatan sulfate in the brain of the subject having Hunter syndrome.

In another aspect, the invention provides a method of increasing degradation of heparan sulfate in the brain of a subject having MPS I or Hunter syndrome, the method comprising administering to the subject a therapeutically effective amount of a pharmaceutical composition of the invention, thereby increasing degradation of heparan sulfate in the brain of the subject having MPS I or Hunter syndrome.

In another aspect, the invention provides a method of increasing degradation of dermatan sulfate in the brain of a subject having MPS I or Hunter syndrome, the method comprising administering to the subject a therapeutically effective amount of a pharmaceutical composition of the invention, thereby increasing degradation of dermatan sulfate in the brain of the subject having MPS I or Hunter syndrome.

In another aspect, the invention provides a method of treating a subject having Fabry disease, the method comprising administering to the subject a therapeutically effective amount of a pharmaceutical composition of the invention wherein the level of glycosphingolipids with α-galactosyl residues, e.g., globotriaosylceramide and related glycosphingolipids, is reduced and/or further accumulation of glycosphingolipids is arrested in the subject, thereby treating the subject having Fabry disease.

In another aspect, the invention provides a method of increasing degradation of glycosphingolipids with α-galactosyl residues, e.g., globotriaosylceramide and related glycosphingolipids, in the brain of a subject having Fabry disease, e.g., in Schwann cells, doral root ganglia and neurons, the method comprising administering to the subject a therapeutically effective amount of a pharmaceutical composition of the invention, thereby increasing degradation of glycosphingolipids with α-galactosyl residues, e.g., globotriaosylceramide, in the brain of the subject having Fabry disease.

In some embodiments, the composition is administered intravenously, intramuscularly, or subcutaneously.

In another aspect, the invention provides a method of producing a composition comprising an enzymatically active human alpha-L-iduronidase (IDUA) protein and a blood-brain barrier carrier peptide (BBB carrier peptide), an enzymatically active human iduronate-2-sulfatase (IDS) protein and a blood-brain barrier carrier peptide (BBB carrier peptide), or an enzymatically active human α-galactosidase A (α-Gal A) protein and a blood-brain barrier carrier peptide (BBB carrier peptide), the method comprising culturing a host cell encoding the human protein under conditions permitting the production of the enzymatically active human IDUA protein, IDS protein, or α-galactosidase A protein, recovering the enzymatically active human protein, and combining the enzymatically active human protein with a blood-brain barrier carrier peptide.

In yet another aspect, the invention provides methods for treating a subject having MPS I. In one embodiment, the MPS I disease is Hurler Syndrome. In another embodiment, the MPS I disease is Scheie Syndrome. In yet another embodiment, the MPS I disease is Hurler-Scheie Syndrome. In yet another embodiment, the invention provides methods for treating a subject having a deficiency in IDUA expression or activity. The methods include comprising administering to the subject (i.e., a subject having MPS I, or in need of such treatment) a therapeutically effective amount of a recombinant human IDUA protein produced by a method described herein, or a therapeutically effective amount of a pharmaceutical composition described herein comprising IDUA and a BBB carrier peptide. Also provided are the use of the pharmaceutical compositions described herein comprising recombinant human IDUA protein and a BBB carrier peptide in the treatment of MPS I in a subject.

In yet another aspect, the invention provides methods for treating a subject having Hunter syndrome. In yet another embodiment, the invention provides methods for treating a subject having a deficiency in IDS expression or activity. The methods include comprising administering to the subject (i.e., a subject having Hunter syndrome, or in need of such treatment) a therapeutically effective amount of a recombinant human IDS protein produced by a method described herein, or a therapeutically effective amount of a pharmaceutical composition described herein comprising IDS and a BBB carrier peptide. Also provided are the use of the pharmaceutical compositions described herein comprising recombinant human IDS protein and a BBB carrier peptide in the treatment of Hunter syndrome in a subject.

In yet another aspect, the invention provides methods for treating a subject having Fabry disease. In yet another embodiment, the invention provides methods for treating a subject having a deficiency in α-galactosidase A expression or activity. The methods include comprising administering to the subject (i.e., a subject having Fabry disease, or in need of such treatment) a therapeutically effective amount of a recombinant human α-galactosidase A protein produced by a method described herein, or a therapeutically effective amount of a pharmaceutical composition described herein comprising α-galactosidase A and a BBB carrier peptide. Also provided are the use of the pharmaceutical compositions described herein comprising recombinant human α-galactosidase A protein and a BBB carrier peptide in the treatment of Fabry disease in a subject.

Unless otherwise defined, all technical and scientific terms used herein have the same meaning as commonly understood by one of ordinary skill in the art to which this invention belongs. Methods and materials are described herein for use in embodiments of the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a bar graph showing levels of heparan sulfate in the brain and liver of wild type (WT) mice administered PBS (control), IDUA knock-out (KO) mice administered PBS (control), IDUA knock-out mice administered IDUA (10 mg/kg), and IDUA knock-out mice administered IDUA (10 mg/kg):K16-ApoE (0.15 mg).

FIG. 2 is a bar graph showing levels of heparan sulfate in the kidneys and heart of wild type (WT) mice administered PBS (control), IDUA knock-out (KO) mice administered PBS (control), IDUA knock-out mice administered IDUA (10 mg/kg), and IDUA knock-out mice administered IDUA (10 mg/kg):K16-ApoE (0.15 mg).

FIG. 3 is bar graphs showing levels of heparan sulfate in the brain and liver of wild type (WT) mice administered PBS (control), IDUA knock-out (KO) mice administered PBS (control), IDUA knock-out mice administered 0.58 mg/kg IDUA (KO IDUA), and IDUA knock-out mice administered 0.58 mg/kg IDUA: 6.5 mg/kg K16-ApoE (KO IDUA:K16). Heparan sulfate levels are shown after 4 weeks of treatment and 8 weeks of treatment.

FIG. 4A is a bar graph showing iduronate-2-sulfatase (IDS) activity in the brain of wild type animals administered PBS (control), IDS (50 mg/kg), or IDS (50 mg/kg):K16 (40 nM). * p<0.05.

FIG. 4B is a bar graph showing iduronate-2-sulfatase (IDS) activity in the liver of wild type animals administered PBS (control), IDS (50 mg/kg), or IDS (50 mg/kg):K16 (40 nM).

FIG. 5 is a bar graph showing levels of heparan sulfate in the brain and liver of wild type (WT) mice administered PBS (control), IDS knock-out (KO) mice administered PBS (control), IDS knock-out mice administered 10 mg/kg IDS (KO 453), IDS knock-out mice administered 1 mg/kg IDS:6.5 mg/kg K16-ApoE (KO IDS (1 mg/kg):K16) and IDS knock-out mice administered 10 mg/kg IDS : 6.5 mg/kg K16-ApoE (KO IDS (10 mg/kg) :K16). Heparan sulfate levels are shown after 4 weeks of treatment.

FIG. 6 is a bar graph showing α-galactosidase A (α-Gal A) activity in the brain (black boxes) and liver (striped boxes) of wild-type (WT) and GLA knock-out (KO) mice administered the indicated treatments: GAL10=α-Gal A (10 mg/kg); GAL10:K16A=α-Gal A (10 mg/kg):K16Apo-E [1:2]; GAL10:K16B=α-Gal A (10 mg/kg):K16Apo-E [1:10].

FIGS. 7A, 7B, 7C, and 7D show α-Gal A levels (FIGS. 7A, 7C) and activity (FIGS. 7B, 7D) in the brain (FIGS. 7A-7B) and liver (FIGS. 7C-7D) of wild-type animals administered PBS (control), α-Gal A (50 mg/kg), or α-Gal A (50 mg/kg):K16 (40 nM). * p<0.05; ** p<0.01.

### DETAILED DESCRIPTION

The instant invention provides methods for delivering therapeutically effective amounts of an enzymatically active human protein (such as an IDUA protein, an IDS protein, or an α-galactosidase A protein) into the brain and central nervous system that is useful to decrease the neurological defects associated with diseases such as MPS I, Hunter syndrome, and/or Fabry disease. Delivery of such therapeutic proteins reduces the risk of cognitive abnormalities, hydrocephalus, hypertrophic cervical pachymeningitis, nerve compression, mental retardation, behavioral abnormalities, cerebral vasculopathy, and stroke.

Enzyme Replacement Therapy for Neurological Disease

The instant invention provides methods of treating human subjects with neurological diseases, such as MPS I, Hunter syndrome, or Fabry disease, by administering a formulation comprising a recombinant human protein (such as IDUA, IDS, or α-galactosidase A), and a carrier peptide that transports the human protein across the blood-brain barrier (BBB), referred to herein as a “BBB carrier peptide.”

In these compositions, the recombinant human protein may be non-covalently complexed with the BBB carrier peptide. As used herein, “complexed with” is meant that the human protein is non-covalently associated with the BBB carrier peptide.

In the present methods and compositions, the human protein is not covalently bound to the BBB carrier peptide. A specific embodiment of the invention provides a composition comprising a human protein and the blood brain barrier peptide of SEQ ID NO:45 (K-K-K-K-K—K-K-K-K-K-K-K-K-K-K-K-L-R-V-R-L-A-S-H-L-R-K-L-R-K-R-L-L-R-D-A). Thus, the BBB carrier peptide is not covalently bound to the human protein, e.g., is not part of a fusion protein with the human protein. BBB carrier peptides can be readily produced by in vitro synthesis, e.g., liquid-phase peptide synthesis or solid-phase peptide synthesis, or alternatively expressed in cells by expression techniques well known in the art and as further described herein.

In some embodiments the human protein (e.g., the IDUA protein, the IDS protein, and the α-Gal A protein) and the BBB carrier peptide can be combined in the desired molar ratio and incubated prior to administration to the patient. In certain embodiments, the human protein and the BBB carrier peptide can be administered to a subject separately in the desired molar ratio either sequentially (e.g., BBB carrier peptide followed by the human protein, or the human protein followed by BBB carrier peptide) or simultaneously (i.e., administering BBB carrier peptide and the human protein to the subject at the same time, without pre-combining or pre-mixing).

In certain embodiments, a molar ratio of the human protein to BBB carrier peptide can range from about 1:1 to about 1:200 (e.g., about 1:2; 1:3; 1:5; 1:8; 1:10; 1:25; 1:30; 1:40; 1:45; 1:50; 1:60; 1:65; 1:70; 1:75; 1:80; 1:90; 1:100; 1:125; 1:135; 1:145; 1:150; 1:160; 1:164, 1:165; 1:166, 1:167; 1:168, 1:169, 1:170; 1:175; 1:180; 1:185; and 1:190). In some embodiments, a molar excess of BBB carrier peptide to the human protein is used. In certain embodiments the molar ratio of the human protein to the BBB carrier peptide is about 1:2 to about 1:10, about 1:10 to about 1:190, about 1:100 to about 1:180, or about 1:155 to about 1:175. In other embodiments, the molar ratio of the human protein to the BBB carrier peptide is about 1:2, about 1:3, about 1:4, about 1:5, about 1:6, about 1:7, about 1:8, about 1:9 or about 1:10. In one embodiment, the molar ratio of the human protein to the BBB carrier peptide is about 1:167.

In further embodiments, the human protein (e.g., the IDUA protein, the IDS protein, and the α-Gal A protein) and BBB carrier peptide are administered in any of the foregoing molar ratios, wherein the human protein is administered at a dose of about 0.2-50 mg of human protein/kg of body weight, e.g., 0.2 mg/kg, 0.3 mg/kg, 0.4 mg/kg, 0.5 mg/kg, 0.58 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, or 50 mg/kg.

In some embodiments, the human protein (e.g., the IDUA protein, the IDS protein, and the α-Gal A protein) and the BBB carrier peptide are administered in any of the foregoing molar ratios, wherein the human protein is administered at a dose of about 0.2-5, 0.5-5, 0.5-4, 0.5-3, 0.5-2, or 0.5-1 mg/kg. In a particular embodiment, the human protein (e.g., the IDUA protein, the IDS protein, and the α-Gal A protein) and the BBB carrier peptide are administered in a molar ratio of about 1:155 to about 1:175 (e.g., about 1:160, 1:161, 1:162, 1:163, 1:164, 1:165, 1:166, 1:167, 1:168, 1:169, 1:170), wherein the human protein administered at a dose of about 0.2-5 mg of IDUA/kg of body weight, e.g., about 0.2 mg/kg, 0.3 mg/kg, 0.4 mg/kg, 0.5 mg/kg, about 0.58 mg/kg, about 1 mg/kg, about 1.5 mg/kg about 2 mg/kg, about 3 mg/kg, about 4 mg/kg or about 5 mg/kg.

In other embodiments, the human protein (e.g., the IDUA protein, the IDS protein, and the α-Gal A protein) and the BBB carrier peptide are administered in a molar ratio of about 1:155 to about 1:175 (e.g., about 1:160, 1:161, 1:162, 1:163, 1:164, 1:165, 1:166, 1:167, 1:168, 1:169, or 1:170), wherein the human protein is administered in an amount effective to reduce, and/or arrest further accumulation of heparan sulfate levels in visceral tissue or urine of a subject.

In some embodiments, the human protein (e.g., the IDUA protein, the IDS protein, and the α-Gal A protein) and the BBB carrier peptide can be combined in the desired mg/kg dose ratio and incubated prior to administration to the patient. In certain embodiments, the human protein (e.g., the IDUA protein, the IDS protein, and the α-Gal A protein) and the BBB carrier peptide can be administered to a subject separately in the desired mg/kg dose ratio either sequentially (e.g., BBB carrier peptide followed by the human protein, or the human protein followed by BBB carrier peptide) or simultaneously (i.e., administering BBB carrier peptide and IDUA, IDS, or α-galactosidase A protein to the subject at the same time, without pre-combining or pre-mixing).

In one embodiment, the human protein (e.g., the IDUA protein, the IDS protein, and the α-Gal A protein) and the BBB carrier peptide are administered in a mg/kg dose ratio of 1:0.5 to about 1:15; about 1:2 to about 1:13; about 1:5 to about 1:9; or about 1:8 to about 1:11. In one embodiment, the human protein (e.g., the IDUA protein, the IDS protein, and the α-Gal A protein) and the BBB carrier peptide are administered in a mg/kg dose ratio of about 1:15, 1:14, 1:13, 1:12, 1:11, 1:10, 1:9, 1:8, 1:7, 1:6, 1:5, 1:4, 1:3, 1:2, 1:1, or 1:0.5.

In further embodiments, the human protein (e.g., the IDUA protein, the IDS protein, and the α-Gal A protein) and BBB carrier peptide are administered in any of the foregoing mg/kg dose ratios, wherein the human protein is administered at a dose of about 0.2-50 mg of human protein/kg of body weight, e.g., 0.2 mg/kg, 0.3 mg/kg, 0.4 mg/kg, 0.5 mg/kg, 0.58 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, or 50 mg/kg.

In some embodiments, the human protein (e.g., the IDUA protein, the IDS protein, and the α-Gal A protein) and the BBB carrier peptide are administered in any of the foregoing mg/kg dose ratios, wherein the human protein is administered at a dose of about 0.2-5, 0.5-5, 0.5-4, 0.5-3, 0.5-2, or 0.5-1 mg/kg. In a particular embodiment, the human protein and the BBB carrier peptide are administered in a mg/kg dose ratio of about 1:6 to about 1:12 (e.g., about 1:7, 1:8, 1:9, 1:10, or 1:11), wherein the human protein is administered at a dose of about 0.2 to about 5 mg of human protein/kg of body weight, e.g., about 0.2 mg/kg, 0.3 mg/kg, 0.4 mg/kg, 0.5 mg/kg, about 0.58 mg/kg, about 1 mg/kg, about 2 mg/kg, about 3 mg/kg, about 4 mg/kg or about 5 mg/kg. In a some embodiments, the human protein (e.g., the IDUA protein, the IDS protein, and the α-Gal A protein) and the BBB carrier peptide are administered in a mg/kg dose ratio of about 1:6 to about 1:12 (e.g., about 1:7, 1:8, 1:9, 1:10, or 1:11), wherein the human protein is administered in an amount effective to reduce, and/or arrest further accumulation of accumulation of heparan sulfate levels in visceral tissue or urine of a subject.

The human protein (e.g., the IDUA protein, the IDS protein, and the α-Gal A protein) and the BBB carrier peptide can be complexed by means known in the art. In certain embodiments, the human protein and the BBB carrier peptide are complexed by combining and incubating the human protein and the BBB carrier peptide at room temperature, e.g., as described in Example 1 for IDUA, e.g., for at least 1 minute, 5 minutes, or 10 minutes, e.g., 10-240 minutes, or 30-120 minutes.

In some embodiments, the methods include administering to the subject a composition comprising a BBB carrier peptide and recombinant human protein in an amount sufficient to treat (e.g., reduce, ameliorate) or prevent one or more aspects of neurological involvement of MPS I. The Examples demonstrate that low dosages of IDUA enzyme (e.g., 10 mg/kg and 0.58 mg/kg) were effective in an in vivo model. Specifically, the Examples demonstrate that a clinically used dosage (0.58 mg/kg) of IDUA formulated with K16-ApoE was effective for delivery of active IDUA to the mammalian brain leading to a dramatic reduction of heparan sulfate. This is surprising, as in other studies performed by the inventors with other enzymes formulated with K16-ApoE, higher enzyme dosages (about 50 mg/kg) were used to achieve detectable levels of enzyme in the brain.

In some embodiments, the methods include administering to the subject a composition comprising a BBB carrier peptide and recombinant human IDS protein in an amount sufficient to treat (e.g., reduce, ameliorate) or prevent one or more aspects of neurological involvement of Hunter syndrome. The Examples demonstrate that low dosages of IDS enzyme (e.g., 10 mg/kg and 50 mg/kg) were effective in an in vivo model. Specifically, the Examples demonstrate that a clinically used dosage (10 mg/kg) of IDS formulated with K16-ApoE was effective for delivery of active IDS to the mammalian brain leading to a dramatic reduction of heparin sulfate.

In some embodiments, the methods include administering to the subject a composition comprising a BBB carrier peptide and recombinant human α-galactosidase A protein in an amount sufficient to treat (e.g., reduce, ameliorate) or prevent one or more aspects of neurological involvement of Fabry disease. The Examples demonstrate that relatively low dosages of α-galactosidase A enzyme (e.g., 50 mg/kg) were effective in an in vivo model. Specifically, the Examples demonstrate that a clinically used dosage (50 mg/kg) of α-galactosidase A formulated with K16-ApoE was effective for delivery of active α-galactosidase A to the mammalian brain. Specifically, the Examples demonstrate that a clinically used dosage (50 mg/kg) of α-galactosidase A formulated with K16-ApoE was effective for delivery of active α-galactosidase A to the mammalian brain and lead to a significant increase in α-galactosidase A enzyme activity.

The pharmaceutical compositions of the invention can be administered therapeutically or prophylactically, or both. The human protein (e.g., the IDUA protein, the IDS protein, and the α-Gal A protein) and the BBB carrier peptide can be administered to the subject, alone or in combination with other therapeutic modalities as known in the art.

The terms “treat,” “treating,” and “treatment” refer to methods of alleviating, abating, or ameliorating a disease or symptom, preventing an additional symptom, ameliorating or preventing an underlying cause of a symptom, inhibiting a disease or condition, arresting the development of a disease or condition, relieving a disease or condition, causing regression of a disease or condition, relieving a condition caused by the disease or condition, or stopping a symptom of the disease or condition either prophylactically and/or after the symptom has occurred.

“Therapeutically effective dose” as used herein refers to the dose (e.g., amount and/or interval) of drug required to produce an intended therapeutic response (e.g., reducing the concentration of heparan sulfate and dermatan sulfate levels, preventing or ameliorating further accumulation of excess heparan sulfate and dermatan sulfate levels, or treatment of cognitive abnormalities, hydrocephalus, hypertrophic cervical pachymeningitis, nerve compression, and/or behavioral abnormalities; e.g., reducing globotriaosylceramide levels, increasing α-Gal A activity in a target tissue, or treatment of cognitive abnormalities, cerebral vasculopathy, stroke, hypohidrosis, dolichoectasia and/or white matter lesions). A therapeutically effective dose refers to a dose that, as compared to a corresponding subject who has not received such a dose, results in improved treatment, healing, prevention (i.e., to reduce risk of or delay onset of disease or disease symptoms), or amelioration of a disease, disorder, or side effect, or a decrease in the rate of the occurrence or advancement of a disease or disorder. The term also includes within its scope doses effective to enhance physiological functions.

As used herein, the term “enzymatically active” refers to a human protein (e.g., the IDUA protein, the IDS protein, and the α-Gal A protein) which includes a fragment of a human protein which participates in an interaction between, for example, an IDUA molecule and a non-IDUA molecule, an IDS molecule and a non-IDS molecule, or an α-galactosidase A molecule and a non-α-galactosidase A molecule, and retains enzyme activity. Alternatively, “enzymatically active” refers to a human protein (e.g., the IDUA protein, the IDS protein, and the α-Gal A protein) which retains one or more activities of the wild-type human protein.

In one embodiment, activities of “enzymatically active” IDUA include, but are not limited to, catalyzing the hydrolysis of unsulfated alpha-L-iduronosidic linkages in dermatan sulfate, catalyzing the hydrolysis of unsulfated alpha-L-iduronosidic linkages in heparan sulfate, or being involved in the degradation of dermatan sulfate and/or heparan sulfate.

In one embodiment, activities of “enzymatically active” IDS include, but are not limited to, catalyzing the hydrolysis of the C2-sulfate ester bond from nonreducing-terminal iduronic acid residues in heparin sulfate, catalyzing the hydrolysis of the C2-sulfate ester bond from nonreducing-terminal iduronic acid residues in dermatan sulfate, or being involved in the degradation of dermatan sulfate and/or heparan sulfate.

In one embodiment, activities of “enzymatically active” α-galactosidase A include, but are not limited to, catabolism of glycosphingolipids, or being involved in the degradation of globotriaosylceramide and related glycosphingolipids or the reduction of globotriaosylceramide and related glycosphingolipids.

Biologically active portions of an IDUA protein include peptides comprising amino acid sequences sufficiently identical to (e.g., at least 90%, 91%, 92%, 93%, 94%, 95%, 96%, 97%, 98%, 99%) or derived from the amino acid sequence of the wild-type human IDUA protein, e.g., the amino acid sequence shown in SEQ ID NO:53, which can include less amino acids than the full length IDUA proteins, and exhibit at least one activity of an IDUA protein described herein. Typically, enzymatically active portions comprise a domain or motif with at least one activity of the IDUA protein, e.g., catalyzing the hydrolysis ofunsulfated alpha-L-iduronosidic linkages in dermatan sulfate, catalyzing the hydrolysis of unsulfated alpha-L-iduronosidic linkages in heparan sulfate, being involved in the degradation of dermatan sulfate, or being involved in the degradation of heparan sulfate.

Biologically active portions of an IDS protein include peptides comprising amino acid sequences sufficiently identical to (e.g., at least 90%, 91%, 92%, 93%, 94%, 95%, 96%, 97%, 98%, 99%) or derived from the amino acid sequence of the wild-type human IDS protein, e.g., the amino acid sequence shown in SEQ ID NO:55, which can include less amino acids than the full length IDS proteins, and exhibit at least one activity of an IDS protein described herein. Typically, enzymatically active portions comprise a domain or motif with at least one activity of the IDS protein, e.g., catalyzing the hydrolysis of the C2-sulfate ester bond from nonreducing-terminal iduronic acid residues in heparin sulfate, catalyzing the hydrolysis of the C2-sulfate ester bond from nonreducing-terminal iduronic acid residues in dermatan sulfate, or being involved in the degradation of dermatan sulfate and/or heparan sulfate.

Biologically active portions of an α-galactosidase A protein include peptides comprising amino acid sequences sufficiently identical to (e.g., at least 90%, 91%, 92%, 93%, 94%, 95%, 96%, 97%, 98%, 99%) or derived from the amino acid sequence of the wild-type human α-galactosidase A protein, e.g., the amino acid sequence shown in SEQ ID NO:56, which can include less amino acids than the full length α-galactosidase A proteins, and exhibit at least one activity of an α-galactosidase A protein described herein. Typically, enzymatically active portions comprise a domain or motif with at least one activity of the α-galactosidase A protein, e.g., catabolism of glycosphingolipids, or being involved in the degradation of globotriaosylceramide and related glycosphingolipids or the reduction of globotriaosylceramide and related glycosphingolipids.

An “isolated” or “purified” protein or biologically active portion thereof is substantially free of cellular material or other contaminating proteins from the cell or tissue source from which the protein was produced, or substantially free from chemical precursors or other chemicals when chemically synthesized. The language “substantially free of cellular material” includes preparations of protein in which the protein is separated from cellular components of the cells from which it is isolated or recombinantly produced.

In one embodiment, the language “substantially free of cellular material” includes preparations of human protein (e.g., IDUA protein, IDS protein, or α-Gal A protein) having less than about 30% (by dry weight) of non-human protein (also referred to herein as a “contaminating protein”), more preferably less than about 20% of non-IDUA, non-IDS, or non-non-αα-galactosidase A protein, still more preferably less than about 10% of non-IDUA, non-IDS, or -galactosidase A protein, and most preferably less than about 5% non-IDUA, non-IDS, or non-α-galactosidase A protein. When the IDUA, IDS, or α-galactosidase A protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations in which the human protein (e.g., the IDUA protein, the IDS protein, or the α-Gal A protein)/BBB carrier peptide composition is separated from chemical precursors or other chemicals which are involved in the synthesis of the protein. In one embodiment, the language “substantially free of chemical precursors or other chemicals” includes preparations of human protein (e.g., the IDUA protein, the IDS protein, or the α-Gal A protein)/BBB carrier peptide composition having less than about 30% (by dry weight) of chemical precursors or non-IDUA, non-IDS, or non-α-galactosidase A chemicals, more preferably less than about 20% chemical precursors or non-IDUA, non-IDS, or non-α-galactosidase A chemicals, still more preferably less than about 10% chemical precursors or non-IDUA, non-IDS, or non-α-galactosidase A chemicals, and most preferably less than about 5% chemical precursors or non-IDUA, non-IDS, or non-α-galactosidase A chemicals.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “subject” or “patient” is intended to include human and non-human animals. Non-human animals include all vertebrates, e.g., mammals and non-mammals, such as non-human primates, sheep, dogs, cats, cows, horses, chickens, amphibians, and reptiles. Preferred subjects include human subjects. In one embodiment, the subject is a human subject having MPS I. In one embodiment, the subject is a human subject having Hurler Syndrome. In another embodiment, the subject is a human subject having Scheie Syndrome. In another embodiment, the subject is a human subject having Hurler-Scheie Syndrome. In another embodiment, the subject is a human subject having a deficiency in IDUA expression or activity. Preferred subjects include human subjects having Hunter syndrome. In another embodiment, the subject is a human subject having a deficiency in IDS expression or activity. Preferred subjects include human subjects having Fabry disease. In another embodiment, the subject is a human subject having a deficiency in α-galactosidase A expression or activity.

Certain embodiments of the invention encompass therapeutic methods that use co-formulations of human protein (e.g., IDUA protein, IDS protein, or α-Gal A protein) and BBB carrier peptides that facilitate the uptake or transport of the recombinant human protein into the pertinent organs and tissues, e.g., into the brain or central nervous system, of a subject.

In one embodiment, the methods of the invention include directly or indirectly delivering the recombinant human IDUA of the invention to the CNS (central nervous system) of a subject for the treatment of MPS I. In one embodiment, the methods of the invention include directly or indirectly delivering the recombinant human IDS of the invention to the CNS (central nervous system) of a subject for the treatment of Hunter syndrome. In one embodiment, the methods of the invention include directly or indirectly delivering the recombinant human α-galactosidase A of the invention to the CNS (central nervous system) of a subject for the treatment of Fabry disease.

For example, the recombinant human protein and BBB carrier peptide may be administered to the patient, e.g., via intravenous (e.g., via intravenous injection or intravenous infusion), intramuscular, subcutaneous, oral, nasal, intranasal, or transdermal administration. In one embodiment, the human protein/BBB carrier peptide composition is administered to the subject intravenously.

The recombinant human protein and BBB carrier peptide can be administered in one or more administrations, applications or dosages. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. In one embodiment, the pharmaceutical compositions can be administered once weekly. In another embodiment, the pharmaceutical compositions can be administered twice weekly. In another embodiment, the pharmaceutical compositions can be administered every other week. In another embodiment, the pharmaceutical compositions can be administered once monthly.

Dosage, toxicity and therapeutic efficacy of the compositions described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and thereby reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of disease manifestation signs) as determined in cell culture. Such information can be used to more accurately determine useful doses in humans. Levels in plasma may be measured, for example, by high performance liquid chromatography.

Exemplary doses include 0.2-50 mg of IDUA/kg of body weight, 0.2-50 mg of IDS/kg of body weight or 0.2-50 mg of α-galactosidase A/kg of body weight, e.g., 0.2 mg/kg, 0.3 mg/kg, 0.4 mg/kg, 0.5 mg/kg, 0.58 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, or 50 mg/kg of body weight. In a particular embodiment, the dosage of IDUA protein is 0.58 mg/kg of body weight. In another embodiment, the dosage of IDUA protein is 10 mg/kg of body weight. In a particular embodiment, the dosage of IDS protein is 10 mg/kg of body weight. In another embodiment, the dosage of IDS protein is 50 mg/kg of body weight. In a particular embodiment, the dosage of α-galactosidase A protein is 10 mg/kg of body weight. In another embodiment, the dosage of α-galactosidase A protein is 50 mg/kg of body weight.

In one aspect, the invention provides methods for increasing hydrolysis of unsulfated alpha-L-iduronosidic linkages in dermatan sulfate and heparan sulfate in the brain of a subject having MPS I by administering to the subject a pharmaceutical composition of the invention. As used herein, a “increasing hydrolysis” or “increased hydrolysis” of unsulfated alpha-L-iduronosidic linkages in dermatan sulfate and heparan sulfate refers to a level of hydrolysis of unsulfated alpha-L-iduronosidic linkages in dermatan sulfate or heparan sulfate that is increased after treatment with a composition comprising IDUA and a BBB carrier peptide, as compared to the level of hydrolysis of unsulfated alpha-L-iduronosidic linkages in dermatan sulfate or heparan sulfate without treatment with the composition comprising IDUA and a BBB carrier peptide, or prior to treatment with the composition comprising IDUA and a BBB carrier peptide.

In one embodiment, the level of hydrolysis of unsulfated alpha-L-iduronosidic linkages in dermatan sulfate and/or heparan sulfate in the brain of a subject having MPS I is increased 50%, 60%, 70%, 80%, or 90% after treatment with a composition of the invention as compared to the level of hydrolysis of unsulfated alpha-L-iduronosidic linkages in dermatan sulfate and heparan sulfate without treatment, or prior to treatment, with the composition. In another embodiment, the level of hydrolysis of unsulfated alpha-L-iduronosidic linkages in dermatan sulfate and heparan sulfate in the brain of a subject having MPS I is increased 2 fold, 3 fold, 4 fold, 5 fold, 6 fold, 7 fold, 8 fold, 9 fold, or 10 fold after treatment with a composition of the invention as compared to the level of hydrolysis of unsulfated alpha-L-iduronosidic linkages in dermatan sulfate and/or heparan sulfate without treatment, or prior to treatment, with the composition. Methods for determining the level of hydrolysis of unsulfated alpha-L-iduronosidic linkages in dermatan sulfate and heparan sulfate are well known in the art. For example, see Warda et al., Glycoconj. J., 2006, 23:555-563; Lawrence et al., Glycobiol., 2004, 14(5):467-479; Shi and Zaia, 2009, J. Biol. Chem., 2009, 284(18):11806-11814; and Ledin et al., J. Biol. Chem., 2004, 279(41):42732-42741.

In one embodiment, the level of hydrolysis of unsulfated alpha-L-iduronosidic linkages in dermatan sulfate and/or heparan sulfate in a subject can, alternatively, be determined by comparison to a “normal” level or a “control” level. The “normal” level of hydrolysis is the level of hydrolysis of the dermatan sulfate and/or heparan sulfate in a subject not afflicted with MPS I. In some embodiments, the “normal” level of hydrolysis may be determined by assessing levels of dermatan sulfate and/or heparan sulfate in a patient sample obtained from a non-MPS I-afflicted patient or from archived patient samples. Alternately, population-average values for normal levels of hydrolysis of unsulfated alpha-L-iduronosidic linkages in dermatan sulfate and/or heparan sulfate may be used.

In another aspect, the invention provides methods for increasing hydrolysis of the C2-sulfate ester bond from nonreducing-terminal iduronic acid residues in heparan sulfate and dermatan sulfate in the brain of a subject having Hunter syndrome by administering to the subject a pharmaceutical composition of the invention. As used herein, a “increasing hydrolysis” or “increased hydrolysis” of the C2-sulfate ester bond from nonreducing-terminal iduronic acid residues in heparan sulfate and dermatan sulfate refers to a level of hydrolysis of the C2-sulfate ester bond from nonreducing-terminal iduronic acid residues in heparan sulfate and dermatan sulfate that is increased after treatment with a composition comprising IDS and a BBB carrier peptide, as compared to the level of hydrolysis of the C2-sulfate ester bond from nonreducing-terminal iduronic acid residues in heparan sulfate and dermatan sulfate without treatment with the composition comprising IDS and a BBB carrier peptide, or prior to treatment with the composition comprising IDS and a BBB carrier peptide.

In one embodiment, the level of hydrolysis of the C2-sulfate ester bond from nonreducing-terminal iduronic acid residues in heparan sulfate and dermatan sulfate in the brain of a subject having Hunter syndrome is increased 50%, 60%, 70%, 80%, or 90% after treatment with a composition of the invention as compared to the level of hydrolysis of the C2-sulfate ester bond from nonreducing-terminal iduronic acid residues in heparan sulfate and dermatan sulfate without treatment, or prior to treatment, with the composition. In another embodiment, the level of hydrolysis of the C2-sulfate ester bond from nonreducing-terminal iduronic acid residues in heparan sulfate and dermatan sulfate in the brain of a subject having Hunter syndrome is increased 2 fold, 3 fold, 4 fold, 5 fold, 6 fold, 7 fold, 8 fold, 9 fold, or 10 fold after treatment with a composition of the invention as compared to the level of hydrolysis of the C2-sulfate ester bond from nonreducing-terminal iduronic acid residues in heparan sulfate and dermatan sulfate without treatment, or prior to treatment, with the composition. Methods for determining the level of hydrolysis of the C2-sulfate ester bond from nonreducing-terminal iduronic acid residues in heparan sulfate and dermatan sulfate are well known in the art.

In another aspect, the invention provides methods for increasing degradation of heparan sulfate and dermatan sulfate in the brain of a subject having MPS I, or in the brain of a subject having Hunter syndrome, by administering to the subject a therapeutically effective amount of the pharmaceutical composition of the invention. In one embodiment, the invention provides methods for increasing degradation of heparan sulfate in the brain of a subject having MPS I, or in the brain of a subject having Hunter syndrome, by administering to the subject a therapeutically effective amount of the pharmaceutical composition of the invention. In one embodiment, the invention provides methods for increasing degradation of dermatan sulfate in the brain of a subject having MPS I, or in the brain of a subject having Hunter syndrome, by administering to the subject a therapeutically effective amount of the pharmaceutical composition of the invention.

As used herein, a “increasing degradation” or “increased degradation” refers to a level of degradation of dermatan sulfate or heparan sulfate that is increased after treatment with a composition comprising IDUA and a BBB carrier peptide, or a composition comprising IDS and a BBB carrier peptide, as compared to the level of degradation of dermatan sulfate or heparan sulfate without treatment, or prior to treatment, with the composition comprising IDUA and a BBB carrier peptide, or the composition comprising IDS and a BBB carrier peptide. In one embodiment, the degradation of both heparan sulfate and dermatan sulfate is increased. In another embodiment, the degradation of heparan sulfate is increased. In another embodiment, the degradation of dermatan sulfate is increased.

In one embodiment, the level of degradation of heparan sulfate and/or dermatan sulfate in the brain of a subject having MPS I or Hunter syndrome is increased 50%, 60%, 70%, 80%, or 90% after treatment with a composition of the invention as compared to the level of degradation of heparan sulfate and/or dermatan sulfate without treatment, or prior to treatment, with the composition. In another embodiment, the level of degradation of heparan sulfate and/or dermatan sulfate in the brain of a subject having MPS I or Hunter syndrome is increased 2 fold, 3 fold, 4 fold, 5 fold, 6 fold, 7 fold, 8 fold, 9 fold, or 10 fold after treatment with a composition of the invention as compared to the level of degradation of heparan sulfate and/or dermatan sulfate without treatment, or prior to treatment, with the composition. In one embodiment, the degradation of both heparan sulfate and dermatan sulfate is increased. In another embodiment, the degradation of heparan sulfate is increased. In another embodiment, the degradation of dermatan sulfate is increased. Methods for determining the level of degradation of heparan sulfate and dermatan sulfate are well known in the art. For example, see Warda et al., Glycoconj. J., 2006, 23:555-563; Lawrence et al., Glycobiol., 2004, 14(5):467-479; Shi and Zaia, 2009, J. Biol. Chem., 2009, 284(18):11806-11814; and Ledin et al., J. Biol. Chem., 2004, 279(41):42732-42741.

In one embodiment, the level of degradation of dermatan sulfate and/or heparan sulfate in a subject can, alternatively, be determined by comparison to a “normal” level or a “control” level. The “normal” level of degradation is the level of degradation of the dermatan sulfate and/or heparan sulfate in a subject not afflicted with MPS I or Hunter syndrome. In some embodiments, the “normal” level of degradation may be determined by assessing levels of dermatan sulfate and/or heparan sulfate in a patient sample obtained from a non-MPS I-afflicted patient, a non-Hunter-syndrome-affected patient or from archived patient samples. Alternately, population-average values for normal levels of degradation of dermatan sulfate and/or heparan sulfate may be used.

In another aspect, the invention provides methods for decreasing levels of heparan sulfate and dermatan sulfate in the brain of a subject having MPS I or Hunter syndrome by administering to the subject a therapeutically effective amount of the pharmaceutical composition of the invention. In one embodiment, the invention provides methods for decreasing levels of heparan sulfate in the brain of a subject having MPS I or Hunter syndrome by administering to the subject a therapeutically effective amount of the pharmaceutical composition of the invention. In one embodiment, the invention provides methods for decreasing levels of dermatan sulfate in the brain of a subject having MPS I or Hunter syndrome by administering to the subject a therapeutically effective amount of the pharmaceutical composition of the invention.

As used herein, a “decreased” or “decreasing” refers to the level of dermatan sulfate or heparan sulfate in the brain of a subject having MPS I after treatment with a composition comprising IDUA and a BBB carrier peptide, as compared to the level of dermatan sulfate or heparan sulfate without treatment, or prior to treatment, with the composition comprising IDUA and a BBB carrier peptide. As used herein, the terms “decreased” or “decreasing” also refer to the level of dermatan sulfate or heparan sulfate in the brain of a subject having Hunter syndrome after treatment with a composition comprising IDS and a BBB carrier peptide, as compared to the level of dermatan sulfate or heparan sulfate without treatment, or prior to treatment, with the composition comprising IDS and a BBB carrier peptide. In one embodiment, the level of both heparan sulfate and dermatan sulfate is decreased. In another embodiment, the level of heparan sulfate is decreased. In another embodiment, the level of dermatan sulfate is decreased.

In one embodiment, the level of heparan sulfate and/or dermatan sulfate in the brain of a subject having MPS I or Hunter syndrome is decreased 50%, 60%, 70%, 80%, or 90% after treatment with a composition of the invention as compared to the level of heparan sulfate and/or dermatan sulfate without treatment, or prior to treatment, with the composition.

In another embodiment, the level of heparan sulfate and/or dermatan sulfate in the brain of a subject having MPS I or Hunter syndrome is decreased 2 fold, 3 fold, 4 fold, 5 fold, 6 fold, 7 fold, 8 fold, 9 fold, or 10 fold after treatment with a composition of the invention as compared to the level of heparan sulfate and/or dermatan sulfate without treatment, or prior to treatment, with the composition. In one embodiment, the levels of both heparan sulfate and dermatan sulfate are decreased. In another embodiment, the level of heparan sulfate is decreased. In another embodiment, the level of dermatan sulfate is decreased. Methods for determining the level of heparan sulfate and dermatan sulfate are well known in the art. For example, see Warda et al., Glycoconj. J., 2006, 23:555-563; Lawrence et al., Glycobiol., 2004, 14(5):467-479; Shi and Zaia, 2009, J. Biol. Chem., 2009, 284(18):11806-11814; and Ledin et al., J. Biol. Chem., 2004, 279(41):42732-42741.

In one embodiment, the level of dermatan sulfate and/or heparan sulfate in a subject can, alternatively, be determined by comparison to a “normal” level or a “control” level. The “normal” level is the level of the dermatan sulfate and/or heparan sulfate in a subject not afflicted with MPS I or Hunter syndrome. In some embodiments, the “normal” level of heparan sulfate or dermatan sulfate may be determined by assessing levels of dermatan sulfate and/or heparan sulfate in a patient sample obtained from a non-MPS I-afflicted patient, a non-Hunter-syndrome-afflicted patient or from archived patient samples. Alternately, population-average values for normal levels of dermatan sulfate and/or heparan sulfate may be used.

BBB Carrier Peptides

Certain embodiments of the invention include compositions that include a carrier peptide that transports the human protein (e.g., the IDUA protein, the IDS protein, or the α-Gal A protein) across the blood-brain barrier (BBB), referred to herein as a BBB carrier peptides. Suitable peptides include those described in Pre-Grant Publication No. US 2012/0107243, the entire disclosure of which is expressly incorporated herein by reference. For example, suitable peptides include a peptide comprising a transferrin-receptor binding site of a transferrin, or a receptor binding domain of an apolipoprotein, e.g., from the receptor binding domain of ApoA, ApoB, ApoC, ApoD, ApoE, ApoE2, ApoE3, and ApoE4, linked to a hydrophilic segment of from 4-50 hydrophilic amino acids chosen from arginine, asparagine, aspartic acid, glutamic acid, glutamine, histidine, lysine, serine, threonine, and tyrosine, or combinations thereof.

In some embodiments, the hydrophilic segment consists of hydrophilic amino acids chosen from lysine or a non-natural lysine derivative, arginine or a non-natural arginine derivative, and combinations thereof. Exemplary sequences include KKKK (SEQ ID NO: 1); KKKKKKKK (SEQ ID NO:2); KKKKKKKKKKKK (SEQ ID NO:3); KKKKKKKKKKKKKKKK (SEQ ID NO:4); RRRR (SEQ ID NO:5); RRRRRRRR (SEQ ID NO:6); RRRRRRRRRRRR (SEQ ID NO:7); RRRRRRRRRRRRRRRR (SEQ ID NO:8); KRKR (SEQ ID NO:9); KKKR (SEQ ID NO:10); KKKRRRKKKRRR (SEQ ID NO:11); and KKKKRRRRKKKKRRRR (SEQ ID NO:12).

In some embodiments, the receptor-binding domain comprises a sequence having at least 80% sequence identity to one of the following sequences:

| SEQ | |
|---|---|
| ID NO: | Sequence |
| 13. | L-R-V-R-L-A-S-H-L-R-K-L-R-K-R-L-L-R-D-A; |
| 14. | S-S-V-I-D-A-L-Q-Y-K-L-E-G-T-T-R-L-T-R-K- |
| | R-G-L- |
| 15. | K-L-A-T-A-L-S-L-S-N-K-F-V-E-G-S-H; |
| 16. | Y-P-A-K-P-E-A-P-G-E-D-A-S-P-E-E-L-S-R-Y- |
| | Y-A-S- |
| 17. | L-R-H-Y-L-N-L-V-T-R-Q-R-Y*; |
| 19. | H-Y-L-N-L-V-T-R-Q-R-Y*; |
| 20. | Y-P-S-D-P-D-N-P-G-E-D-A-P-A-E-D-L-A-R-Y- |
| | Y-S-A- |
| 21. | L-R-H-Y-I-N-L-I-T-R-Q-R-Y*; |
| 22. | A-P-L-E-P-V-Y-P-G-D-D-A-T-P-E-Q-M-A-Q-Y- |
| | A-A-E- |
| 23. | L-R-R-Y-I-N-M-L-T-R-P-R-Y*, |
| 24. | L-R-S-R-L-A-S-H-L-R-K-L-R-K-R-L-L-R-D-A |
| 25. | L-R-V-R-M-A-S-H-L-R-K-L-R-K-R-L-L-R-D-A |
| 26. | L-R-V-R-L-A-T-H-L-R-K-L-R-K-R-L-L-R-D-A |
| 27. | L-R-V-R-L-A-S-H-L-R-K-L-P-K-R-L-L-R-D-A |
| 28. | L-R-V-R-L-A-S-H-L-R-K-L-R-K-R-L-M-R-D-A |
| 29. | L-R-V-R-L-A-S-H-L-R-N-L-R-K-R-L-L-R-D-A |
| 30. | L-R-V-R-L-A-S-H-L-R-K-V-R-K-R-L-L-R-D-A |
| 31. | L-R-V-R-M-S-S-H-L-R-K-L-R-K-R-L-L-R-D-A |
| 32. | L-R-V-R-L-A-S-H-L-R-N-V-R-K-R-L-L-R-D-A |
| 33. | L-R-V-R-L-A-S-H-L-R-N-M-R-K-R-L-L-R-D-A |
| 34. | L-R-A-R-M-A-S-H-L-R-K-L-R-K-R-L-L-R-D-A |
| 35. | L-R-V-R-L-S-S-H-L-R-K-L-R-K-R-L-M-R-D-A |
| 36. | L-R-S-R-L-A-S-H-L-R-K-L-R-K-R-L-M-R-D-A |
| 37. | L-R-V-R-L-S-S-H-L-P-K-L-R-K-R-L-L-R-D-A |
| 38. | L-R-V-R-L-A-S-H-L-R-K-M-R-K-R-L-M-R-D-A |
| 39. | L-R-V-R-L-A-S-H-L-R-N-L-P-K-R-L-L-R-D-A |
| 40. | L-R-L-R-L-A-S-H-L-R-K-L-R-K-R-L-L-R-D-L |
| 41. | L-R-V-R-L-A-N-H-L-R-K-L-R-K-R-L-L-R-D-L |

In the above, Y* is tyrosine or a tyrosine derivative (e.g., an amidated tyrosine). See, e.g., Ballantyne, G. H., Obesity Surgery, 16:651-658 2006.

In some embodiments, the BBB carrier peptide comprises a sequence having at least 80% sequence identity to one of the following sequences:

| | |
|---|---|
| 42. | K-K-K-K-L-R-V-R-L-A-S-H-L-R-K-L-R-K-R-L-L- |
| | R-D-A; |
| 43. | K-K-K-K-K-K-K-K-L-R-V-R-L-A-S-H-L-R-K-L-R- |
| | K-R-L-L-R-D-A |
| 44. | K-K-K-K-K-K-K-K-K-K-K-K-L-R-V-R-L-A-S-H-L- |
| | R-K-L-R-K-R-L-L-R-D-A |
| 45. | K-K-K-K-K-K-K-K-K-K-K-K-K-K-K-K-L-R-V-R-L- |
| | A-S-H-L-R-K-L-R-K-R-L-L-R-D-A |
| 46. | K-K-K-K-K-K-K-K-K-K-K-K-K-K-K-K-K-K-K-K-L- |
| | R-V-R-L-A-S-H-L-R-K-L-R-K-R-L-L-R-D-A |
| 47. | K-K-K-K-S-S-V-I-D-A-L-Q-Y-K-L-E-G-T-T-R-L- |
| | T-R-K-R-G-L-K-L-A-T-A-L-S-L-S-N-K-F-V-E-G- |
| | S-H |
| 48. | K-K-K-K-K-K-K-K-S-S-V-I-D-A-L-Q-Y-K-L-E-G- |
| | T-T-R-L-T-R-K-R-G-L-K-L-A-T-A-L-S-L-S-N-K- |
| | F-V-E-G-S-H |
| 49. | K-K-K-K-K-K-K-K-K-K-K-K-S-S-V-I-D-A-L-Q-Y- |
| | K-L-E-G-T-T-R-L-T-R-K-R-G-L-K-L-A-T-A-L-S- |
| | L-S-N-K-F-V-E-G-S-H |
| 50. | K-K-K-K-K-K-K-K-K-K-K-K-K-K-K-K-S-S-V-I-D- |
| | A-L-Q-Y-K-L-E-G-T-T-R-L-T-R-K-R-G-L-K-L-A- |
| | T-A-L-S-L-S-N-K-F-V-E-G-S-H |
| 51. | K-K-K-K-K-K-K-K-K-K-K-K-K-K-K-K-K-K-K-K-S- |
| | S-V-I-D-A-L-Q-Y-K-L-E-G-T-T-R-L-T-R-K-R-G- |
| | L-K-L-A-T-A-L-S-L-S-N-K-F-V-E-G-S-H |

In some embodiments, the BBB carrier peptide comprises the peptide K16ApoE, i.e., K-K-K-K—K-K-K-K-K-K-K-K-K-K-K-K-L-R-V-R-L-A-S-H-L-R-K-L-R-K-R-L-L-R-D-A (SEQ ID NO:45). In some embodiments, the BBB carrier peptide is L-R-K-L-R-K-R-L-L-R-L-R-K-L-R—K-R-L-L-R (SEQ ID NO:52). In some embodiments, the BBB carrier peptide is not L-R-K-L-R-K-R-L-L-R-L-R-K-L-R-K-R-L-L-R (SEQ ID NO:52).

In one embodiment, the carrier peptide that facilitates the human protein (e.g., the IDUA protein, the IDS protein, or the α-galactosidase A protein) crossing of the blood-brain barrier (BBB) is at least 80% identical to any one of SEQ ID NOs:13-51. In another embodiment, the carrier peptide that facilitates crossing of the IDUA, IDS, or α-Gal A across the blood-brain barrier (BBB) is at least 85% identical to any one of SEQ ID NOs:13-51. In another embodiment, the carrier peptide that facilitates crossing of the IDUA, IDS, or α-Gal A across the blood-brain barrier (BBB) is at least 90% identical to any one of SEQ ID NOs:13-51. In another embodiment, the carrier peptide that facilitates crossing of the IDUA, IDS, or α-Gal A across the blood-brain barrier (BBB) is at least 95% identical to any one of SEQ ID NOs:13-51. In yet another embodiment, the carrier peptide that facilitates crossing of the IDUA, IDS, or α-Gal A across the blood-brain barrier (BBB) is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs:13-51.

In one embodiment, the carrier peptide that facilitates crossing of the human protein (e.g., the IDUA protein, the IDS protein, or the α-galactosidase A protein) across the blood-brain barrier (BBB) is at least 80% identical to SEQ ID NO:45. In another embodiment, the carrier peptide that facilitates crossing of the IDUA, IDS, or α-Gal A across the blood-brain barrier (BBB) is at least 85% identical to SEQ ID NO:45. In another embodiment, the carrier peptide that facilitates crossing of the IDUA, IDS, or α-Gal A across the blood-brain barrier (BBB) is at least 90% identical to SEQ ID NO:45. In another embodiment, the carrier peptide that facilitates crossing of the IDUA, IDS, or α-Gal A across the blood-brain barrier (BBB) is at least 95% identical to SEQ ID NO:45. In yet another embodiment, the carrier peptide that facilitates crossing of the IDUA, IDS, or α-Gal A across the blood-brain barrier (BBB) is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:45.

“Percent sequence identity” refers to the degree of sequence identity between any two or more sequences. The sequence to be compared typically has a length that is from 80 percent to 200 percent of the length of a reference sequence (e.g., 82, 85, 87, 89, 90, 93, 95, 97, 99, 100, 105, 110, 115, 120, 130, 140, 150, 160, 170, 180, 190, or 200 percent of the length of the reference sequence). A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). Chema et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10; gap extension penalty: 5.0; and weight transitions: yes. For fast pairwise alignment of peptide sequences, the following parameters are used: word size: 1; window size: 5; scoring method: percentage; number of top diagonals: 5; gap penalty: 3. For multiple alignment of peptide sequences, the following parameters are used: weight matrix: blosum; gap opening penalty: 10.; gap extension penalty: 0.5; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run from several online sources.

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

In some embodiments, the BBB carrier peptide is modified, e.g., is amidated at the N-terminus (e.g., during a synthesis reaction).

Pharmaceutical Compositions

The compositions comprising a human protein (e.g., an IDUA protein, an IDS protein, or an α-galactosidase A protein) and BBB carrier peptide can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interference with their activity. In some embodiments, a water-soluble carrier suitable for intravenous administration is used.

The composition or medicament, if desired, can also contain minor amounts of wetting or emulsifying agents, or pH buffering agents. The composition can be a liquid solution, suspension, emulsion, tablet, pill, capsule, sustained release formulation, or powder. The composition can also be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The composition or medicament can be formulated in accordance with the routine procedures as a pharmaceutical composition adapted for administration to human beings. For example, in some embodiments, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

Recombinant Human IDUA, IDS, and α-Gal A

The present compositions and methods use a recombinant human IDUA enzyme with the same amino acid sequence as the native enzyme. Amino acid sequences of human IDUA are available in GenBank at Acc. No. NP_000194. An exemplary human IDUA sequence is as follows:

| | |
|---|---|
| 1 | mrplrpraal lallasllaa ppvapaeaph lvhvdaaral wplrrfwrst gfcpplphsq |
| 61 | adqyvlswdq qlnlayvgav phrgikqvrt hwllelvttr gstgrglsyn fthldgyldl |
| 121 | lrenqllpgf elmgsasghf tdfedkqqvf ewkdlvssla rrylgrygla hvskwnfetw |
| 181 | nepdhhdfdn vsmtmqgfln yydacseglr aaspalrlgg pgdsfhtppr splswgllrh |
| 241 | chdgtnfftg eagvrldyis ihrkgarssi silegekvva qqirqlfpkf adtpiyndea |
| 301 | dplvgwslpq pwradvtyaa mvvkvlaqhq nlllanttsa fpyallsndn aflsyhphpf |
| 361 | aqrtltarfq vnntrpphvq llrkpvitam gllalldeeq lwaevsgagt vldsnhtvgv |
| 421 | lasahrpqgp adawraavli yasddtrahp nrsvavtlrl rgvppgpglv yvtryldngl |
| 481 | cspdgewrrl grpvfptaeq frrmraaedp vaaaprplpa ggrltlrpal rlpslllvhv |
| 541 | carpekppgq vtrlralplt qgqlvlvwsd ehvgskclwt yeiqfsqdgk aytpvsrkps |
| 601 | tfnlfvfspd tgaysgsyry raldywarpg pfsdpvpyle vpvprgppsp gnp |

In one embodiment, the IDUA protein comprises SEQ ID NO:53. In another embodiment, the IDUA protein consists of SEQ ID NO:53. In one embodiment, the IDUA protein is at least 80% identical to SEQ ID NO:53. In another embodiment, the IDUA protein is at least 85% identical to SEQ ID NO:53. In another embodiment, the IDUA protein is at least 90% identical to SEQ ID NO:53. In another embodiment, the IDUA protein is at least 95% identical to SEQ ID NO:53. In another embodiment, the IDUA protein is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:53.

In some embodiments, the IDUA enzyme used in the methods and compositions described herein comprise the above SEQ ID NO:53 without amino acids 1-19 (signal sequence; underlined above), i.e., comprises amino acids 20-653 of SEQ ID NO:53. Signal sequences appropriate for expression systems commonly used to support clinical and commercial amounts of protein are well known in the art. In one embodiment, the IDUA protein comprises amino acids 20-653 of SEQ ID NO:53. In another embodiment, the IDUA protein consists of amino acids 20-653 of SEQ ID NO:53. In another embodiment the IDUA protein is not a fusion protein. In one embodiment, the IDUA protein is at least 80% identical to amino acids 20-653 of SEQ ID NO:53. In another embodiment, the IDUA protein is at least 85% identical to amino acids 20-653 of SEQ ID NO:53. In another embodiment, the IDUA protein is at least 90% identical to amino acids 20-653 of SEQ ID NO:53. In another embodiment, the IDUA protein is at least 95% identical to amino acids 20-653 of SEQ ID NO:53. In another embodiment, the IDUA protein is at least 80%, 81%, 82%, 83%, 84%, 85%, 86%, 87%, 88%, 89%, 90%, 91%, 92%, 93%, 94%, 95%, 96%, 97%, 98%, or 99% identical to amino acids 20-653 of SEQ ID NO:53.

Other IDUA proteins are well known in the art and are described in, for example, U.S. Pat. No. 6,426,208, the entire contents of which are expressly incorporated herein by reference. Methods for expressing IDUA proteins are also commonly known in the art and are described in, for example, U.S. Pat. No. 6,426,208, the entire contents of which are expressly incorporated herein by reference.

Biologically active portions of an IDUA protein include peptides comprising amino acid sequences sufficiently identical to (e.g., at least 90%, 91%, 92%, 93%, 94%, 95%, 96%, 97%, 98%, 99%) or derived from the amino acid sequence of the wild-type human IDUA protein, e.g., the amino acid sequence shown in SEQ ID NO:53, which can include less amino acids than the full length IDUA proteins, and exhibit at least one activity of an IDUA protein described herein. Typically, enzymatically active portions comprise a domain or motif with at least one activity of the IDUA protein, e.g., catalyzing the hydrolysis of unsulfated alpha-L-iduronosidic linkages in dermatan sulfate and heparan sulfate, being involved in the degradation of dermatan sulfate, or being involved in the degradation of heparan sulfate.

In one embodiment, the invention provides variants of IDUA having increased stability as compared to wild-type IDUA. In another embodiment, the invention provides variants of IDUA having decreased immunogenicity as compared to wild-type IDUA. In yet another embodiment, the invention provides variants of IDUA having increased catalytic activity as compared to wild-type IDUA.

In some embodiments, a nucleic acid sequence encoding IDUA as described in the present application, can be molecularly cloned (inserted) into a suitable vector for propagation or expression in a suitable expression system, including cultured cells. An exemplary nucleic acid sequence is in GenBank at NM_000203. An exemplary human IDUA nucleic acid sequence is as follows:

(SEQ ID NO: 54)
atgcgtcccctgcgcccccgcgccgcgctgctggcgctcctggcctc
gctcctggccgcgcccccggtggccccggccgaggccccgcacctgg
tgcatgtggacgcggcccgcgcgctgtggcccctgcggcgcttctgg
aggagcacaggcttctgccccccgctgccacacagccaggctgacca
gtacgtcctcagctgggaccagcagctcaacctcgcctatgtgggcg
ccgtccctcaccgcggcatcaagcaggtccggacccactggctgctg
gagcttgtcaccaccagggggtccactggacggggcctgagctacaa
cttcacccacctggacgggtacctggaccttctcagggagaaccagc
tcctcccagggtttgagctgatgggcagcgcctcgggccacttcact
gactttgaggacaagcagcaggtgtttgagtggaaggacttggtctc
cagcctggccaggagatacatcggtaggtacggactggcgcatgttt
ccaagtggaacttcgagacgtggaatgagccagaccaccacgacttt
gacaacgtctccatgaccatgcaaggcttcctgaactactacgatgc
ctgctcggagggtctgcgcgccgccagccccgccctgcggctgggag
gccccggcgactccttccacaccccaccgcgatccccgctgagctgg
ggcctcctgcgccactgccacgacggtaccaacttcttcactgggga
ggcgggcgtgcggctggactacatctccctccacaggaagggtgcgc
gcagctccatctccatcctggagcaggagaaggtcgtcgcgcagcag
atccggcagctcttccccaagttcgcggacacccccatttacaacga
cgaggcggacccgctggtgggctggtccctgccacagccgtggaggg
cggacgtgacctacgcggccatggtggtgaaggtcatcgcgcagcat
cagaacctgctactggccaacaccacctccgccttcccctacgcgct
cctgagcaacgacaatgccttcctgagctaccacccgcaccccttcg
cgcagcgcacgctcaccgcgcgcttccaggtcaacaacacccgcccg
ccgcacgtgcagctgttgcgcaagccggtgctcacggccatggggct
gctggcgctgctggatgaggagcagctctgggccgaagtgtcgcagg
ccgggaccgtcctggacagcaaccacacggtgggcgtcctggccagc
gcccaccgcccccagggcccggccgacgcctggcgcgccgcggtgct
gatctacgcgagcgacgacacccgcgcccaccccaaccgcagcgtcg
cggtgaccctgcggctgcgcggggtgccccccggcccgggcctggtc
tacgtcacgcgctacctggacaacgggctctgcagccccgacggcga
gtggcggcgcctgggccggcccgtcttccccacggcagagcagttcc
ggcgcatgcgcgcggctgaggacccggtggccgcggcgccccgcccc
ttacccgccggcggccgcctgaccctgcgccccgcgctgcggctgcc
gtcgcttttgctggtgcacgtgtgtgcgcgccccgagaagccgcccg
ggcaggtcacgcggctccgcgccctgcccctgacccaagggcagctg
gttctggtctggtcggatgaacacgtgggctccaagtgcctgtggac
atacgagatccagttctctcaggacggtaaggcgtacaccccggtca
gcaggaagccatcgaccttcaacctctttgtgttcagcccagacaca
ggtgctgtctctggctcctaccgagttcgagccctggactactgggc
ccgaccaggccccttctcggaccctgtgccgtacctggaggtccctg
tgccaagagggcccccatccccgggcaatccatga

The present compositions and methods also use a recombinant human IDS enzyme with the same amino acid sequence as the native enzyme. Amino acid sequences of human IDS are available in GenBank at Acc. No. NP_000193.1, NP_001160022.1, and NP_006114.1. See also U.S. Pat. No. 5,932,211 and Wilson et al., Proc Natl Acad Sci USA. November 1990; 87(21): 8531-8535. An exemplary human sequence is as follows:

| (SEQ ID NO: 55) | | |
|---|---|---|
| 1 | MPPPRTGRGL LWLGLVLSSV CVALGSETQA NSTTDALNVL LIIVDDLRPS LGCYGDKLVR | |
| 61 | SPNIDQLASH SLLFQNAFAQ QAVCAPSRVS FLTGRRPDTT RLYDFNSYWR VHAGNFSTIP | |
| 121 | QYFKENGYVT MSVGKVFHPG ISSNHTDDSP YSWSFPPYHP SSEKYENTKT CRGPDGELHA | 181 |
| | NLLCPVDVLD VPEGTLPDKQ STEQAIQLLE KMKTSASPFF LAVGYHKPHI PFRYPKEFQK | 241 |
| | LYPLENITLA PDPEVPDGLP PVAYNPWMDI RQREDVQALN ISVPYGPIPV DFQRKIRQSY | |
| 301 | FASVSYLDTQ VGRLLSALDD LQLANSTIIA FTSDHGWALG EHGEWAKYSN FDVATHVPLI | 361 |
| | FYVPGRTASL PEAGEKLFPY LDPFDSASQL MEPGRQSMDL VELVSLFPTL AGLAGLQVPP | 421 |
| | RCPVPSFHVE LCREGKNLLK HFRFRDLEED PYLPGNPREL IAYSQYPRPS DIPQWNSDKP | 481 |
| | SLKDIKIMGY SIRTIDYRYT VWVGFNPDEF LANFSDIHAG ELYFVDSDPL QDHNMYNDSQ | 541 |
| | GGDLFQLLMP | |

In some embodiments, the IDS enzyme used in the methods and compositions described herein comprise the above SEQ ID NO:55 without amino acids 1-25 (signal sequence; underlined above), i.e., comprises amino acids 26-550 of SEQ ID NO:55. Signal sequences appropriate for expression systems commonly used to support clinical and commercial amounts of protein are well known in the art.

A nucleic acid sequence encoding IDS as described in the present application, can be molecularly cloned (inserted) into a suitable vector for propagation or expression in a suitable expression system, including transgenic animals and cultured cells. An exemplary nucleic acid sequence is in GenBank at NM_000202.6, NM_001166550.2, and NM_006123.4. See also U.S. Pat. No. 5,932,211 and U.S. Pat. No. 6,541,254.

The present compositions and methods also use a recombinant human α-galactosidase A enzyme with the same amino acid sequence as the native enzyme. An amino acid sequence of human α-galactosidase A is available in GenBank at Acc. No. NP_000160.1. See also WO2008128089). An exemplary human sequence is as follows:

| (SEQ ID NO: 56) | |
|---|---|
| 1 | MQLRNPELHL GCALALRFLA LVSWDIPGAR ALDNGLARTP TMGWLHWERF MCNLDCQEEP |
| 61 | DSCISEKLFM EMAELMVSEG WKDAGYEYLC IDDCWMAPQR DSEGRLQADP QRFPHGIRQL |
| 121 | ANYVHSKGLK LGIYADVGNK TCAGFPGSFG YYDIDAQTFA DWGVDLLKFD GCYCDSLENL |
| 181 | ADGYKHMSLA LNRTGRSIVY SCEWPLYMWP FQKPNYTEIR QYCNHWRNFA DIDDSWKSIK |
| 241 | SILDWTSFNQ ERIVDVAGPG GWNDPDMLVI GNFGLSWNQQ VTQMALWAIM AAPLFMSNDL |
| 301 | RHISPQAKAL LQDKDVIAIN QDPLGKQGYQ LRQGDNFEVW ERPLSGLAWA VAMINRQEIG |
| 361 | GPRSYTIAVA SLGKGVACNP ACFITQLLPV KRKLGFYEWT SRLRSHINPT GTVLLQLENT |
| 421 | MQMSLKDLL |

In some embodiments, the α-Gal A enzyme used in the methods and compositions described herein comprise the above SEQ ID NO:56 without amino acids 1-31 (signal sequence; underlined above), i.e., comprises amino acids 32-429 of SEQ ID NO:56. Signal sequences appropriate for expression systems commonly used to support clinical and commercial amounts of protein are well known in the art.

A nucleic acid sequence encoding α-Gal A as described in the present application, can be molecularly cloned (inserted) into a suitable vector for propagation or expression in a suitable expression system, including transgenic animals and cultured cells. An exemplary nucleic acid sequence is in GenBank at NM_000169.2. See also WO2008128089.

A wide variety of expression vectors can be used to practice the present invention, including, without limitation, a prokaryotic expression vector; a yeast expression vector; an insect expression vector, an avian expression vector and a mammalian expression vector.

Exemplary vectors suitable for the present invention include, but are not limited to, viral based vectors (e.g., AAV based vectors, retrovirus based vectors, and plasmid based vectors). Typically, a nucleic acid encoding the human protein is operably linked to various regulatory sequences or elements.

In some embodiments, the recombinant IDUA, IDS, or α-Gal A used in the formulations and methods described herein is produced in vitro using cultured host cells, which in particular embodiments are suitable for producing IDUA, IDS, or α-Gal A at a large scale. Suitable host cells can be derived from a variety of organisms, including, but not limited to, mammals, plants, birds (e.g., avian systems), insects, yeast, and bacteria. In some embodiments, host cells are mammalian cells.

Any mammalian cell or cell type conducive to cell culture, and to expression of polypeptides, may be utilized in accordance with the present invention as a host cell. Non-limiting examples of mammalian cells that may be used in accordance with the present invention include human embryonic kidney 293 cells (HEK293), HeLa cells; BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells+/−DHFR (CHO, Urlaub and Chasin, Proc. Natl. Acad. Sci. USA, 77:4216 (1980), see also U.S. Pat. No. 5,356,804); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al, Annals N.Y. Acad. Sci., 383:44-68 (1982)); MRC 5 cells; FS4 cells; and a human hepatoma line (Hep G2).

Any non-mammalian derived cell or cell type susceptible to cell culture, and to expression of polypeptides, may be utilized in accordance with the present invention as a host cell, provided the IDUA, IDS, or α-Gal A is modified with appropriate oligosaccharides for facilitating uptake into affected cells and targeting to lysosomes. Glycosylation of IDUA, IDS, and α-Gal A have been well studied and methods for detecting oligosaccharides on proteins are well known in the art (see, e.g., Millat et al. Biochem J. Aug. 15, 1997; 326 (Pt 1): 243-247; Lee, Glycobiology, 13(4):305-13 (2003); See also U.S. Pat. No. 5,932,211 and U.S. Pat. No. 6,541,254). Such glycosylation can occur either within the cell or post-expression using oligosaccharide remodeling techniques well known in the art. Non-limiting examples of non-mammalian host cells and cell lines that may be used in accordance with the present invention include cells and cell lines derived from Pichia pastoris, Pichia methanolica, Pichia angusta, Schizosacccharomyces pombe, Saccharomyces cerevisiae, and Yarrowia lipolytica for yeast; Sodoptera frugiperda, Trichoplusis ni, Drosophila melangoster and Manduca sexta for insects; and Escherichia coli, Salmonella typhimurium, Bacillus subtilis, Bacillus licheniformis, Bacteroides fragilis, Clostridia perfringens, Clostridia difficile for bacteria; and Xenopus Laevis from amphibian.

Various cell culture media and conditions may be used to produce the human protein (e.g., an IDUA protein, an IDS protein, or an α-galactosidase A protein). For example, IDUA, IDS, or α-Gal A may be produced in serum-containing or serum-free medium. In some embodiments, a recombinant IDUA, IDS, or α-Gal A is produced in serum-free medium. In some embodiments, a recombinant IDUA, IDS, or α-Gal A is produced in an animal free medium, i.e., a medium that lacks animal-derived components. In some embodiments, a recombinant IDUA, IDS, or α-Gal A is produced in a chemically defined medium. As used herein, the term “chemically-defined nutrient medium” refers to a medium of which substantially all of the chemical components are known. In some embodiments, a chemically defined nutrient medium is free of animal-derived components such as serum, serum derived proteins (e.g., albumin or fetuin), and other components. In some cases, a chemically defined medium comprises one or more proteins (e.g., protein growth factors or cytokines). In some cases, a chemically defined nutrient medium comprises one or more protein hydrolysates. In other cases, a chemically defined nutrient medium is a protein-free media, i.e., a serum-free media that contains no proteins, hydrolysates or components of unknown composition.

In some embodiments, a chemically defined medium may be supplemented by one or more animal derived components. Such animal derived components include, but are not limited to, fetal calf serum, horse serum, goat serum, donkey serum, human serum, and serum derived proteins such as albumins (e.g., bovine serum albumin or human serum albumin). While the addition of serum is desirable because it contains constituents, such as vitamins, amino acids, growth factors, and hormones, it also constitutes a concentrated source of exogenous protein, which can impede recombinant protein purification. Thus, in some embodiments, a suitable medium is a xeno-free media, e.g., a medium that does not contain any bovine serum or bovine serum derived components. For example, a xeno-free medium may contain one or more of human serum albumin, human transferrin, human insulin, and human lipids. In some embodiments, a suitable medium contains fetuin-depleted serum. Fetuin may be depleted from serum using various methods known in the art. For example, fetuin may be depleted from serum by antibody affinity chromatography. (See, e.g., Toroian D and Price P A, Calcif Tissue Int (2008) 82:116-126). In some embodiments, a suitable medium is fetuin-free.

Various cell culture conditions may be used to produce recombinant lysosomal enzyme proteins at large scale including, but not limited to, roller bottle cultures, bioreactor batch cultures and bioreactor fed-batch cultures. In some embodiments, IDUA, IDS, or α-Gal A is produced by cells cultured in suspensions. In some embodiments, IDUA, IDS, or α-Gal A is produced by adherent cells.

In some embodiments, the recombinant human protein used in the formulations and methods described herein is produced in transgenic poultry. Transgenic poultry have been developed that express exogenous protein and lay eggs containing the exogenous protein; these birds are an ideal “bioreactor” for production of human proteins for, for example, IDS replacement therapy for Hunter syndrome, α-Gal A replacement therapy for Fabry disease, or IDUA replacement therapy for MPS I. See, e.g., US Pub. No. 2014/0065690.

Transgenic poultry useful in methods described herein can be made by any method known in the art. For example, germ-line transgenic chickens may be produced by injecting replication-defective retrovirus into the subgerminal cavity of chick blastoderms in freshly laid eggs (U.S. Pat. Nos. 5,162,215 and 6,397,777; Bosselman et al., Science 243:533-534 (1989); Thoraval et al., Transgenic Research 4:369-36 (1995)). Alternatively, a transgene can be microinjected into the germinal disc of a fertilized egg to produce a stable transgenic founder bird that passes the gene to the F1 generation (Love et al. Bio/Technology 12:60-63 (1994)). In preferred embodiments, the transgene is introduced by a replication-deficient retroviral vector, e.g., as described in U.S. Pat. Nos. 5,162,215, 7,521,591 or 7,524,626, or in USPG Pub. No. 20090307786, and 20090193534; additional exemplary methodologies for expressing proteins, including lysosomal acid lipases, in avian expression systems are described in PCT Publication WO 2004/015123 and U.S. Pub. Nos. 20060191026, 20090178147; 20090180989; 20100083389; and 2010033219, 20130209436, 20130095092, or 20140044697; the entire disclosures of all of the foregoing are incorporated herein by reference.

“Poultry” refers to avians (birds) that can be kept as livestock, including but not limited to, chickens, duck, turkey, quail and ratites. The term “poultry derived” or “avian derived” refers to a composition or substance produced by or obtained from poultry. For example, “poultry derived” may refer to chicken derived, turkey derived and/or quail derived.

Various methods may be used to purify or isolate the human protein (e.g., IDUA, IDS or α-galactosidase A) produced according to various methods described herein. In some embodiments, IDUA, IDS or α-galactosidase A is secreted into the medium and thus cells and other solids may be removed, as by centrifugation or filtering for example, as a first step in the purification process. Alternatively or additionally, the IDUA, IDS or α-galactosidase A is bound to the surface of the host cell. In this embodiment, the host cells expressing the polypeptide or protein are lysed for purification. Lysis of mammalian host cells can be achieved by any number of means well known to those of ordinary skill in the art, including physical disruption by glass beads and exposure to high pH conditions.

The IDUA, IDS or α-galactosidase A may be isolated and purified by standard methods including, but not limited to, chromatography (e.g., ion exchange, affinity, size exclusion, and hydroxyapatite chromatography), gel filtration, centrifugation, or differential solubility, ethanol precipitation or by any other available technique for the purification of proteins (See, e.g., U.S. Pat. No. 5,356,804; Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference). For immunoaffinity chromatography in particular, IDUA, IDS or α-galactosidase A may be isolated by binding it to an affinity column comprising antibodies that were raised against that protein and were affixed to a stationary support. Alternatively, affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin or aprotinin may be added at any or all stages in order to reduce or eliminate degradation of the polypeptide or protein during the purification process. Protease inhibitors are particularly desired when cells must be lysed in order to isolate and purify the expressed IDUA, IDS or α-galactosidase A.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Examples

The invention is further described in the following examples, which do not limit the scope of the invention described in the claims.

### Example 1. Biodistribution of IDUA and K16Apo-E (SEQ ID NO: 45)

This example describes an experiment performed to determine the biodistribution and efficacy of IDUA following two intravenous infusions in IDUA knock-out mice. Six C57/BL6 wild-type mice (4 on study) and 16 IDUA knock-out mice (12 on study) that were 12-13 weeks of age and about 30 g in weight were used. Mice were sedated under isoflurane anesthesia during the five minute tail vein infusion (500 μL/mouse) according to the below Table 1.

TABLE 1

| | | | | IDUA | K16Apo-E | |
|---|---|---|---|---|---|---|
| | | | | Nominal | Construct | Dosing |
| Group | N | Mice | Compound | Dose/Mouse | ND/Mouse | Regimen |
| 1 | 4 | C57B/L6 | PBS control | 0 | 0 | IV tail vein |
| | | | | | | Day 1 and Day 4 |
| 2 | 4 | IDUA | PBS control | 0 | 0 | IV tail vein |
| | | knock-out | | | | Day 1 and Day 4 |
| 3 | 4 | IDUA | IDUA | 0.3 mg | 0 | IV tail vein |
| | | knock-out | (10 mg/kg) | (3.6 nmole) | | Day 1 and Day 4 |
| 4 | 4 | IDUA | IDUA | 0.3 mg | 0.15 mg | IV tail vein |
| | | knock-out | (10 mg/kg)/ | (3.6 nmole) | (35 nmole) | Day 1 and Day 4 |
| | | | K16Apo-E | | | |
| | | | (6.5 mg/kg) | | | |

In order to create the IDUA/K16Apo-E co-formulation, IDUA (72.6 kDa) and K16-ApoE (4.52 kDa) were mixed by gentle vortexing and stood at room temperature for fifteen minutes. The 500 μL dose was administered via a 500 cc insulin syringe with 10 small boluses of about 500 μL each, with a 30 second gap in between each bolus. Animals were dosed via tail vein infusion once on Day 1 and once on Day 4 (total of 2 doses). Non-fasted animals were used for this study.

Blood was collected from all animals on Days -2 and Day 7. On Day 3, 100 μL of whole blood was collected via submandibular vein into EDTA containing collection tubes. Samples were gently inverted three times, centrifuged at 3500 rpm at 2-8° C. for 15 minutes, and processed within 30 minutes of collection. Collected plasma (about 50 μL) was placed into silanized polypropylene tubes containing about 5 μL ProBlock protease inhibitor and stored on dry ice, then moved to −70° C. until analysis.

On Day 7, blood was collected via cardiac puncture and about 500 μL was placed in a K2 EDTA anticoagulant tube and was inverted gently three times. Samples were centrifuged at 3500 rpm at 2-8° C. for 15 minutes, and processed within 30 minutes of collection. Plasma was divided into two 120 μL aliquots in silanized polypropylene tubes, each containing about 10 μL ProBlock protease inhibitor and stored on dry ice, then moved to −70° C. until analysis.

Animals were euthanized 72 hours after the second intravenous injection. Following euthanasia (via CO₂ asphyxiation) and a terminal cardiac puncture blood collection; animals were perfused with PBS. Brain, liver, heart, spleen, kidneys and lungs were collected, rinsed briefly with PBS, and split/saved as follows.

1. Brain
a. One lobe was snap frozen in silanized polypropylene tubes
b. One lobe was sectioned so anatomical structures are preserved in both slices
   i. One section was preserved in formalin
   ii. One section was embedded in OCT medium

2. Liver
a. Left lobe was snap frozen in silanized polypropylene tubes (pre-weighed and containing 5 uL of inhibitor)
b. Right lobe was weighed and frozen in 2 mL tube

3. Heart
a. Half of the heart was weighed and snap frozen in 2 mL tube
b. The other half was snap frozen in silanized polypropylene tubes

4. Spleen
a. One third was snap frozen in silanized polypropylene tubes
b. One third was weighed and frozen in a 2 mL tube
c. One third was preserved in formalin medium

5. Kidneys
a. One kidney was snap frozen in silanized polypropylene tubes
b. Half of the second kidney was weighed and frozen in a 2 mL tube c. The other half of the second kidney was preserved on formalin medium 6. Lungs
a. One lung was snap frozen in silanized polypropylene tubes
b. Half of the second lung was weighed and frozen in a 2 mL tube
c. The other half of the second lung was preserved in formalin medium

Assessment of IDUA levels and activity in plasma and tissues was determined. The ability of K16-ApoE technology to increase human IDUA enzyme uptake and improve enzyme replacement therapy (ERT) efficacy was assessed by measuring heparan sulfate levels, which is one of the substrates that accumulates in tissues in Hurler Syndrome in the MPS I knock-out mouse model. Methods for measuring heparan sulfate levels from murine and rodent tissues are known in the art and described in, for example, Warda et al., Glycoconj. J., 2006, 23:555-563; Lawrence et al., Glycobiol., 2004, 14(5):467-479; Shi and Zaia, 2009, J. Biol. Chem., 2009, 284(18):11806-11814 and Ledin et al., J. Biol. Chem., 2004, 279(41):42732-42741.

Liver and Brain

The ability of K16-ApoE IDUA to increase human IDUA enzyme uptake across the blood brain barrier was assessed by measuring heparan sulfate levels in the brain. Liver tissue was used as a control.

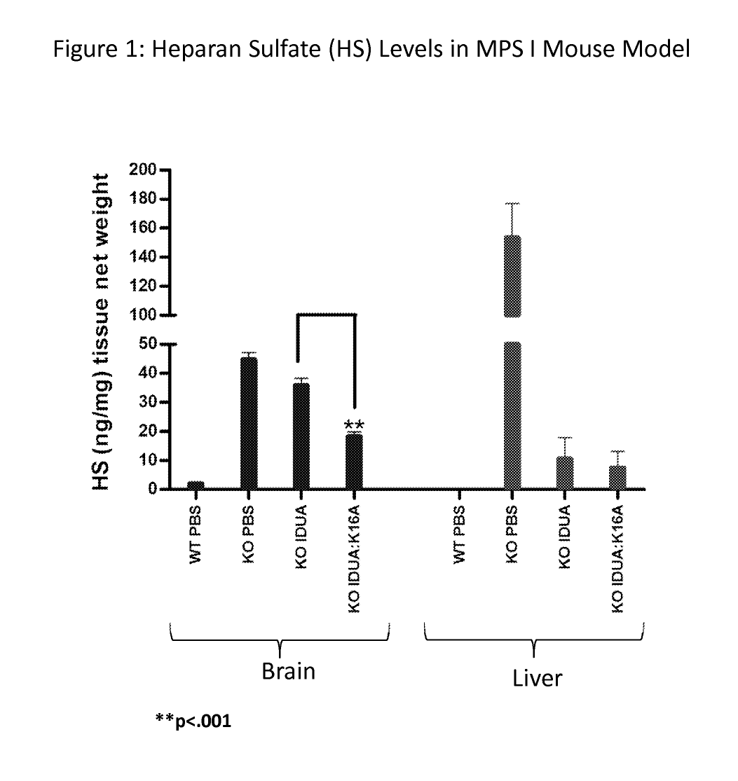

As expected, knock-out animals show dramatic accumulation of heparan sulfate (HS) in the brain and liver compared to wild-type animals (see FIG. 1; knock-out (KO) PBS control bars). Treatment with human IDUA enzyme at a dosage of 10 mg/kg with a two dose regimen separated by three days did not significantly affect the accumulation of heparan sulfate in the brains of the knock-out animals. The same treatment, however, resulted in a dramatic reduction in heparan sulfate in the liver. This was expected, as it has previously been shown that IDUA cannot cross the blood-brain barrier.

Surprisingly, however, addition of 35 nmole K16-ApoE to the 10 mg/kg IDUA dose (with a final molar ratio of IDUA to K16-ApoE peptide of 1:10) led to a very significant reduction in the heparan sulfate levels in the brain as compared to the treatment with IDUA alone (see FIG. 1).

Addition of the K16-ApoE peptide to the IDUA treatment did not affect the levels of heparan sulfate in the liver versus treatment alone. The liver heparan sulfate levels also serve as a control to make sure that the animals administered K16-ApoE did not inadvertently get more IDUA than the animals administered IDUA alone.

These results indicate that administration of IDUA formulated with K16-ApoE results in delivery of active IDUA to the mammalian brain.

Kidney and Heart

The ability of K16-ApoE IDUA to increase human IDUA enzyme uptake in the heart and kidneys was assessed by measuring heparan sulfate levels, which is one of the substrates that accumulates in tissues in Hurler Syndrome in the MPS I knock-out mouse model.

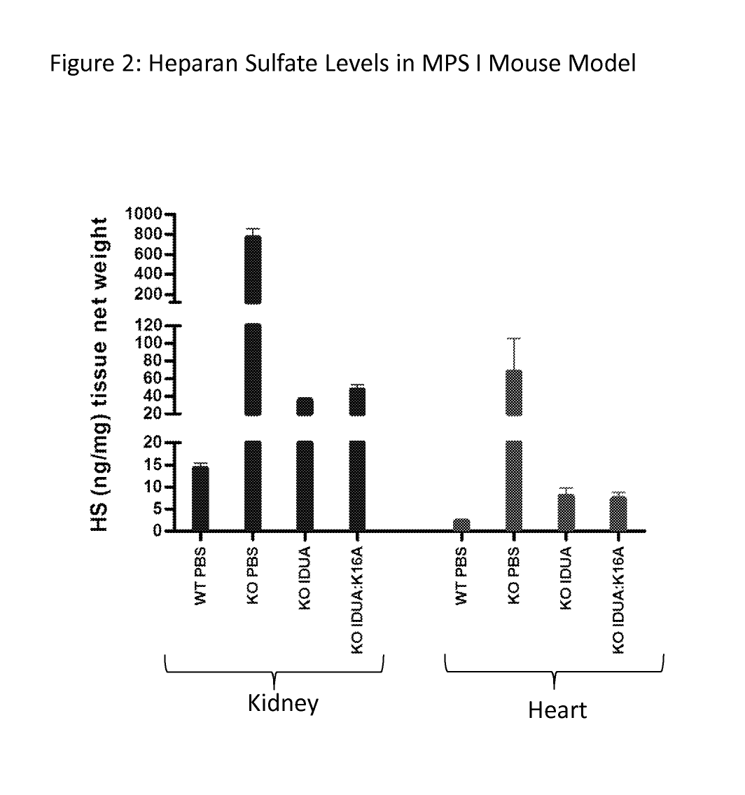

As expected, knock-out animals show dramatic accumulation of heparan sulfate (HS) in the kidney and heart compared to wild-type animals (see FIG. 2; knock-out (KO) PBS control bars). Treatment with human IDUA enzyme at a dosage of 10 mg/kg with a two dose regimen separated by three days resulted in a dramatic reduction in heparan sulfate in the both the kidneys and the heart. Moreover, addition of 35 nmole K16-ApoE to the 10 mg/kg IDUA dose (with a final molar ratio of IDUA to K16-ApoE peptide of 1:10) also led to a very significant reduction in the heparan sulfate levels in the kidney and heart as compared to the treatment with the PBS control (see FIG. 2).

IDUA treatment lead to significant reduction of heparan sulfate levels versus untreated knock-out animal controls in the kidneys and heart, and the addition of K16-ApoE peptide did not affect the accumulation of heparan sulfate in the heart or kidneys versus treatment with IDUA alone.

These results indicate that administration of IDUA formulated with K16-ApoE results in delivery of active IDUA to the mammalian kidneys and heart. The delivery of active IDUA formulated with K16-ApoE to the mammalian kidneys and heart is surprising, as it was previously unknown whether the K16-ApoE composition would interact with the IDUA peptide in other tissues.

The low dosage (10 mg/kg) at which IDUA formulated with K16-ApoE was effective for delivery of active IDUA to the mammalian brain.

### Example 2. Biodistribution of IDUA and K16Apo-E (SEQ ID NO: 45)

This example describes an experiment performed to determine the biodistrubtion and efficacy of IDUA following chronic low dose infusions in IDUA knock-out mice. Ten C57/BL6 wild-type mice and 30 IDUA knock-out mice that were 12-13 weeks of age and about 30 g in weight were used. Mice were sedated under isoflurane anesthesia during the five minute tail vein infusion (200 μL/mouse) according to the below Table 2.

| | | | | IDUA | K16Apo-E | | |
|---|---|---|---|---|---|---|---|
| | | | | Nominal | Construct | Dosing | |
| Group | N | Mice | Compound | Dose/Mouse | ND/Mouse | Regimen | Take down |
| 1 | 10 | C57B/L6 | PBS control | 0 | 0 | IV tail | 4 animals after 4 |
| | | | | | | vein | weeks and 6 animals |
| | | | | | | Weekly | at 8 weeks post |
| | | | | | | | dosing |
| 2 | 10 | IDUA | PBS control | 0 | 0 | IV tail | 4 animals after 4 |
| | | knock-out | | | | vein | weeks and 6 animals |
| | | | | | | Weekly | at 8 weeks post |
| | | | | | | | dosing |
| 3 | 10 | IDUA | IDUA | 0.0174 mg | 0 | IV tail | 4 animals after 4 |
| | | knock-out | (0.58 mg/kg) | (0.21 nmole) | | vein | weeks and 6 animals |
| | | | | | | Weekly | at 8 weeks post |
| | | | | | | | dosing |
| 4 | 10 | IDUA | IDUA | 0.0174 mg | 0.15 mg | IV tail | 4 animals after 4 |
| | | knock-out | (0.58 mg/kg)/ | (0.21 nmole) | (35 nmole) | vein | weeks and 6 animals |
| | | | K16Apo-E | | | Weekly | at 8 weeks post |
| | | | (6.5 mg/kg) | | | | dosing |

All animals were taken down 48 hours after the last dose. In order to create the IDUA/K16Apo-E co-formulation, IDUA and K16-ApoE were mixed by gentle vortexing and stood at room temperature for fifteen minutes. The 200 μL dose was administered via a 500 cc insulin syringe as a slow bolus infusion. Animals were dosed via tail vein infusion once weekly. A subgroup of animals (4 animals) was taken down approximately 4 weeks after study initiation (5 injections total) and 48 hours after the last injection. The remaining group (6 animals) was taken down approximately 8 weeks after study initiation; 48 hours after the last injection (9 injections total). Non-fasted animals were used for this study.

Blood was collected from all animals on Days −2 and Day 27, and Day 58. Blood collected on Day −2 and Day 27 via submandibular vein into EDTA containing collection tubes. Samples were gently inverted three times, centrifuged at 3500 rpm at 2-8° C. for 15 minutes, and processed within 30 minutes of collection. Collected plasma (about 50 μL) was placed into silanized polypropylene tubes containing about 5 μL ProBlock protease inhibitor and stored on dry ice, then moved to −70° C. until analysis. On Day 58, blood was collected via cardiac puncture and about 500 μL was placed in a K2 EDTA anticoagulant tube and was inverted gently three times. Samples were centrifuged at 3500 rpm at 2-8° C. for 15 minutes, and processed within 30 minutes of collection. Plasma was divided into two 120 μL aliquots in silanized polypropylene tubes, each containing about 10 μL ProBlock protease inhibitor and stored on dry ice, then moved to −70° C. until analysis.

Animals were euthanized 48 hours after the last intravenous injection they received (Day 29 or Day 58). Following euthanasia (via CO₂ asphyxiation) and a terminal cardiac puncture blood collection; animals were perfused with PBS. Brain, liver, heart, spleen, kidneys and lungs were collected, rinsed briefly with PBS, and split/saved as follows.

1. Brain
a. One lobe was snap frozen in silanized polypropylene tubes
b. One lobe was sectioned so anatomical structures are preserved in both slices
   i. One section was preserved in formalin
   ii. One section was embedded in OCT medium

2. Liver
a. Left lobe was snap frozen in silanized polypropylene tubes (pre-weighed and containing 5 uL of inhibitor)
b. Right lobe was weighed and frozen in 2 mL tube

3. Heart
a. Half of the heart was weighed and snap frozen in 2 mL tube
b. The other half was snap frozen in silanized polypropylene tubes

4. Spleen
a. One third was snap frozen in silanized polypropylene tubes
b. One third was weighed and frozen in a 2 mL tube
c. One third was preserved in formalin medium

5. Kidneys
a. One kidney was snap frozen in silanized polypropylene tubes
b. Half of the second kidney was weighed and frozen in a 2 mL tube
c. The other half of the second kidney was preserved on formalin medium

6. Lungs
a. One lung was snap frozen in silanized polypropylene tubes
b. Half of the second lung was weighed and frozen in a 2 mL tube
c. The other half of the second lung was preserved in formalin medium

Assessment of IDUA levels and activity in tissues was determined as described in Example 1.

Liver and Brain

The ability of K16-ApoE IDUA to increase human IDUA enzyme uptake across the blood brain barrier was assessed by measuring heparan sulfate levels in the brain. Liver tissue was used as a control.

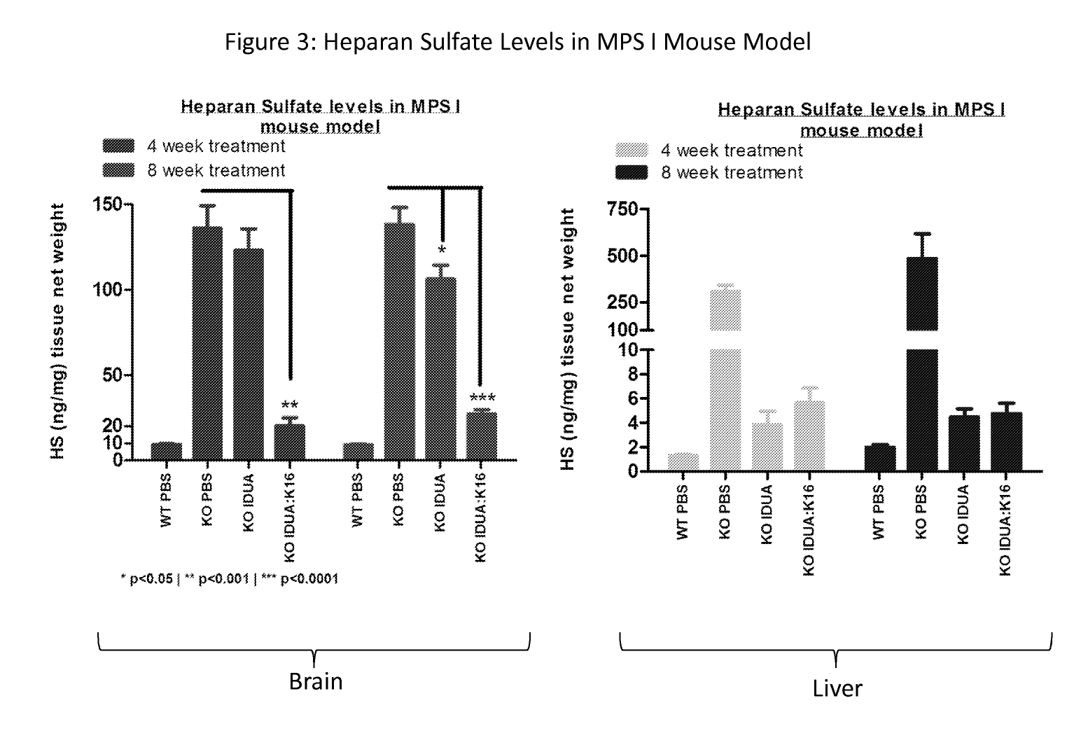

As expected, knock-out animals show dramatic accumulation of heparan sulfate (HS) in the brain and liver compared to wild-type animals (see FIG. 3; knock-out (KO) PBS control bars). Treatment with human IDUA enzyme at a dosage of 0.58 mg/kg with a once weekly dose regimen (i.e., the prescribed dose regimen for laronidase, the regulatory-approved form of IDUA indicated for patients with MPS I) did not significantly affect the accumulation of heparan sulfate in the brains of the knock-out animals after 4 weeks (5 doses) of treatment. This was expected, as it has previously been shown that IDUA cannot cross the blood-brain barrier. The same treatment, however, resulted in a dramatic reduction in heparan sulfate in the liver.

Surprisingly, however, addition of 35 nmole K16-ApoE to the 0.58 mg/kg IDUA dose (with a final molar ratio of IDUA to K16-ApoE peptide of 1:167) led to a very significant reduction in the heparan sulfate levels in the brain as compared to the treatment with IDUA alone (see FIG. 3). Treatment with human IDUA enzyme at a dosage of 0.58 mg/kg with a once weekly dose regimen for 8 weeks led to a modest but significant reduction in the levels of heparan sulfate in the brain, which is presumably due to clearance of heparan substrate from the endothelial cells lining the brain, as IDUA is not known to cross the blood brain barrier. The same treatment, however, resulted in a much more dramatic reduction in heparan sulfate levels in the brain when 35 nmole K16-ApoE was added to the 0.58 mg/kg IDUA dose (see FIG. 3). The same treatment resulted in reduction in heparan sulfate in the liver with no difference among the IDUA vs IDUA: K16-ApoE as expected.

Addition of the K16-ApoE peptide to the IDUA treatment did not affect the levels of heparan sulfate in the liver versus IDUA treatment alone. The liver heparan sulfate levels also serve as a control to make sure that the animals administered K16-ApoE—did not inadvertently get more IDUA than the animals administered IDUA alone.

These results indicate that administration of IDUA formulated with K16-ApoE results in delivery of active IDUA to the mammalian brain.

This is the first report that shows that a low, clinically used dosage (0.58 mg/kg) of IDUA formulated with K16-ApoE was effective for delivery of active IDUA to the mammalian brain leading to a dramatic reduction of heparan sulfate. This is surprising, as in other studies performed by the inventors with other enzymes formulated with K16-ApoE, higher enzyme dosages of about 50 mg/kg were used to achieve detectable levels of enzyme in the brain.

### Example 3. Biodistribution of IDS and K16Apo-E

This example describes the results of experiments in which IDS (obtained from a commercial source) was mixed with a BBB carrier peptide K16Apo-E and administered by intravenous injection in the tail vein of wild-type mice with slow bolus. Male mice, 15-17 weeks of age, were treated according to the following Table 3.

TABLE 3

| | | | | IDS | K16Apo-E/ |
|---|---|---|---|---|---|
| Group | N | Mice | Description | Dose/mouse | mouse |
| 1 | 3 | C57B/L6 | saline control | 0 | 0 |
| | | | (PBS) | | |
| 2 | 3 | C57B/L6 | IDS alone | 1,150.00 ug | 0 |
| | | | (50 mg/kg) | | |
| 3 | 3 | C57B/L6 | IDS (50 mg/kg | 1,150.00 ug | 181 ug |
| | | | [11.8 nmole]): | | |
| | | | 40 nmole | | |
| | | | K16-ApoE | | |

The treatment mixtures in Table 3 were prepared about 1 hour before injection at room temperature, and subjected to slow vortex (no bubbles) for a few seconds at 15 minute intervals prior to injection (vortexed 4×). Respective components were aliquoted as needed by AM body weight measurement. Final volume for injection for each mouse was normalized to 575 uL with sterile PBS. Non-fasted animals were used for this study. Mice were dosed at a pace of 2 mice every 15 minutes based on necropsy timing to ensure tissues were collected ˜24 hrs post dose.

Mice were sedated under isoflurane anesthesia during the 5 minute tail vein dosing (575 uL/mouse). The 575 uL dose was administered via an insulin syringe by slow injection over about three to five minutes. Two of the three animals recovered normally; one showed signs of lethargy and was monitored for 1 hour.

Animals were euthanized 24 hours after intravenous injection. Following euthanasia (via CO₂ asphyxiation) and a terminal cardiac puncture blood collection; animals were perfused with PBS. Brain and liver were collected, rinsed briefly with PBS, and split/saved as follows.

1. Brain
a. One lobe was snap frozen in silanized polypropylene tubes (pre-weighed and containing 5 uL of inhibitor)
b. One lobe was sectioned so anatomical structures are preserved in both slices
   i. One section was preserved in formalin
   ii. One section was embedded in OCT medium

2. Liver
a. Left lobe was snap frozen in silanized polypropylene tubes (pre-weighed and containing 5 uL of inhibitor)
b. Right lobe was split with one half preserved in formalin and one half embedded in OCT medium (laid flat)

Blood was collected via cardiac puncture and ˜500 uL and placed in a K2 EDTA anticoagulant tube (containing ˜10 uL ProBlock protease inhibitor) and inverted gently three times. Samples were centrifuged @ 3500 RPM, 2-8° C. for 15 minutes (process within 30 minutes of collection). Plasma was aliquoted at 120 uL into duplicate silanized polypropylene tubes at stored on dry ice and then moved to −70° C. until analysis.

Statistical Analysis of Numerical in-Life Data was Assessed Using GraphPad Prism Software.

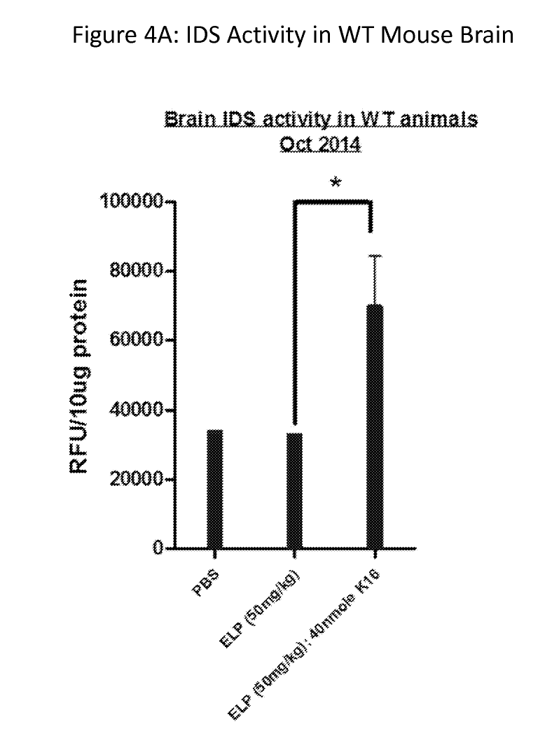

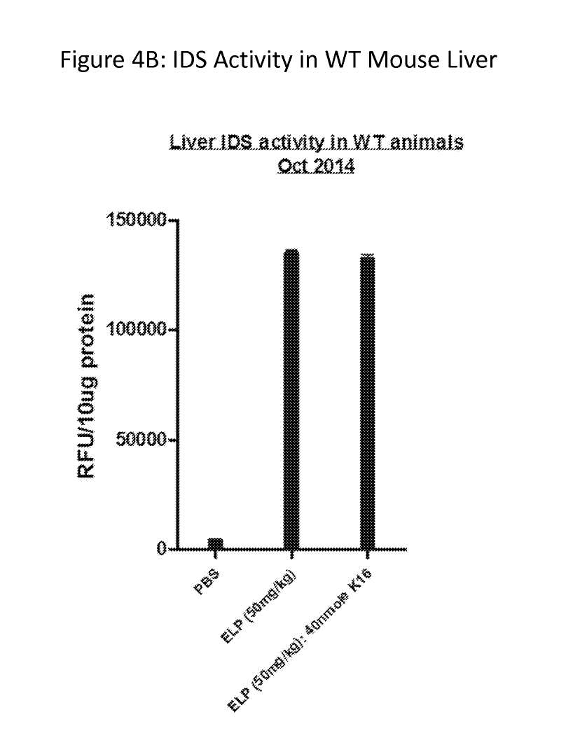

IDS activity in WT animals was determined substantially as described in Voznyi et al., J Inherit Metab Dis. 2001 November; 24(6):675-80, and showed an increase in the brain when mice were injected with 50 mg/kg IDS (FIG. 1A). A mixture of IDS:K16-ApoE at a 1:2.6 molar ratio showed a very significant enhancement of IDS enzyme brain penetration (FIG. 4A). K16-ApoE led to approximately twofold increase in IDS enzyme activity in the brain when included in the mixture prior to injection (FIG. 4A). Liver IDS levels and activity did not differ significantly with the addition of K16-ApoE (FIG. 4B), indicating that the increase in brain activity and levels with the peptide was not due to technical issues with the injection. If anything, IDS activity in the liver tended to be somewhat lower in mice treated with the IDS:K16-ApoE mixture, possibly due to a shift in biodistribution of the injected enzyme.

These results indicate that administration of IDS formulated with K16-ApoE results in delivery of active IDS to the mammalian brain.

### Example 4. Biodistribution of IDS and K16Apo-E (SEQ ID NO: 45) in MPS II Model

This example describes an experiment performed to determine the biodistrubtion and efficacy of IDS following five weekly intravenous infusions for four weeks in IDS knock-out mice in an MPS II model. Mice were sedated under isoflurane anesthesia during the five minute tail vein infusion (200 μL/mouse) according to the below Table 4.

TABLE 4

| Group | Description |
|---|---|
| 1. | WT saline control |
| 2. | IDS KO saline control |
| 3. | IDS KO SBC453 (1 mg/kg) |
| 4. | IDS KO SBC453 (1 mg/kg):K16ApoE (6.5 mg/kg) |
| 5. | IDS KO SBC453 (10 mg/kg) |

Animals were euthanized 24 hours after the last intravenous injection they received (Day 29). Following euthanasia (via CO₂ asphyxiation) and a terminal cardiac puncture blood collection; animals were perfused with PBS. Brain and liver were collected, rinsed briefly with PBS, and split/saved as follows.

1. Brain
a. One lobe was snap frozen in silanized polypropylene tubes
b. One lobe was sectioned so anatomical structures are preserved in both slices
   i. One section was preserved in formalin
   ii. One section was embedded in OCT medium

2. Liver
a. Left lobe was snap frozen in silanized polypropylene tubes (pre-weighed and containing 5 uL of inhibitor)
b. Right lobe was weighed and frozen in 2 mL tube

Assessment of IDS levels and activity in tissues was determined as described in Example 3.

Liver and Brain

The ability of K16-ApoE IDS to increase human IDS enzyme uptake across the blood brain barrier was assessed by measuring heparan sulfate levels in the brain. Liver tissue was used as a control.

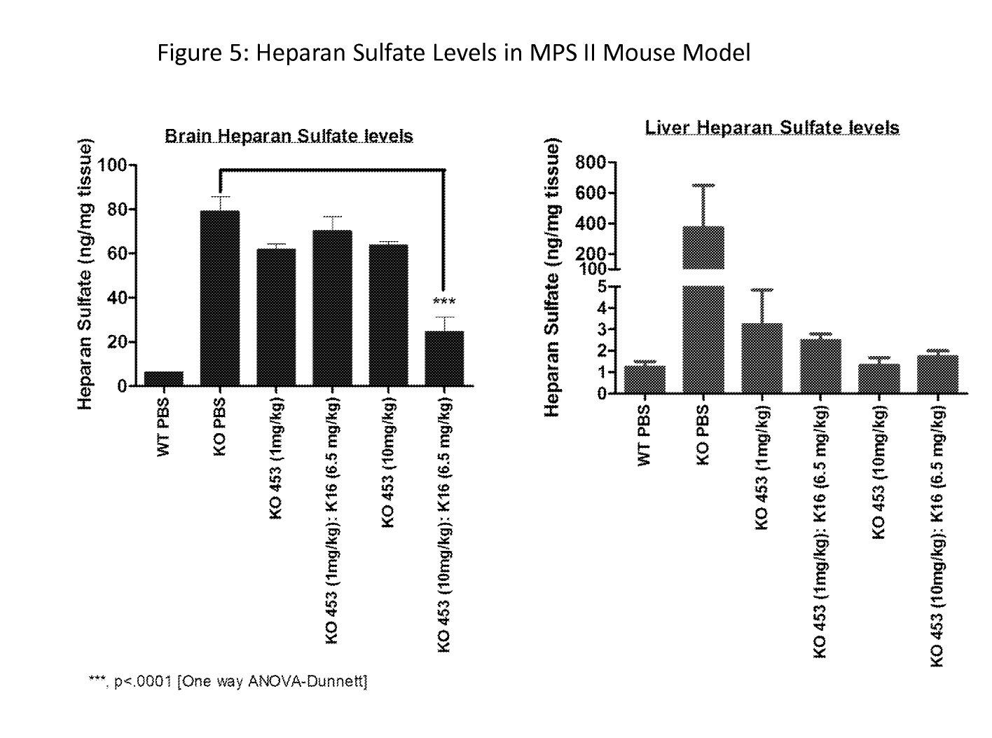

As expected, knock-out animals show dramatic accumulation of heparan sulfate (HS) in the brain and liver compared to wild-type animals (see FIG. 5; knock-out (KO) PBS control bars). Treatment with human IDS enzyme at a dosage of 1 mg/kg or 10 mg/kg with a once weekly dose regimen did not significantly affect the accumulation of heparan sulfate in the brains of the knock-out animals after 4 weeks (5 doses) of treatment. The same treatment, however, resulted in a dramatic reduction in heparan sulfate in the liver.

Surprisingly, however, addition of K16-ApoE to the 10 mg/kg IDS dose led to a significant reduction in the heparan sulfate levels in the brain as compared to the treatment with IDS alone (see FIG. 5). The same treatment resulted in reduction in heparan sulfate in the liver with no difference among the IDS vs IDS: K16-ApoE as expected. Addition of the K16-ApoE peptide to the IDS treatment did not affect the levels of heparan sulfate in the liver versus IDS treatment alone. The liver heparan sulfate levels also serve as a control to make sure that the animals administered K16-ApoE—did not inadvertently get more IDS than the animals administered IDS alone.

These results indicate that administration of IDS formulated with K16-ApoE results in delivery of active IDS to the mammalian brain.

This is the first report that shows that a relatively low, clinically used dosage (10 mg/kg) of IDS formulated with K16-ApoE was effective for delivery of active IDS to the mammalian brain leading to a dramatic reduction of heparan sulfate.

### Example 5. Biodistribution of α-Gal A and K16Apo-E—Study 1

This example describes the results of experiments in which α-Gal A (obtained from a commercial source) was mixed with a BBB carrier peptide K16Apo-E and administered intravenously to mice that lack the α-Gal A gene (GLA KO mice; described in Ohshima et al., Proc. Natl. Acad. Sci. USA, 94:2540-2544 (1997)) and to wild type mice. Male mice, 15-17 weeks of age, were treated according to the following Table 5A:

TABLE 5A

| | No. of | | | α-Gal A | K16Apo-E |
|---|---|---|---|---|---|
| Group | Males | Mice | Treatment | Dose/mouse | Dose/mouse |
| 1 | 4 | C57/B16 WT | PBS control | — | — |
| 2 | 4 | GLA KO | PBS control | — | — |
| 3 | 4 | GLA KO | α-Gal A alone (10 mg/kg) | 300 ug | 0 |
| 4 | 4 | GLA KO | α-Gal A 1:2 K16Apo-E | 300 ug | 27.9 ug |
| | | | (10 mg/kg:930 ug/kg) | | |
| 5 | 4 | GLA KO | α-Gal A 1:10 K16Apo-E | 300 ug | 139.5 ug |
| | | | (10 mg/kg:4.65 mg/kg) | | |

The treatment mixtures in Table 5A were prepared about 1 hour before injection at room temperature, and subjected to slow vortex (no bubbles) for a few seconds at 15 minute intervals prior to injection (vortexed 4×). Respective components were aliquoted as needed by AM body weight measurement. Final volume for injection for each mouse was normalized to 200 uL with sterile PBS. Non-fasted animals were used for this study. Mice were dosed at a pace of 2 mice every 15 minutes based on necropsy timing to ensure tissues were collected ˜24 hrs post dose.

Mice were sedated under isoflurane anesthesia during the 5 minute tail vein dosing (200 uL/mouse). The 200 uL dose was administered via a 500 cc insulin syringe with 5 small boluses of ˜40 uL each with a 45 second gap in between each bolus. The animals recovered normally with no signs of lethargy.

Assessment of α-Gal A levels in plasma and tissues was determined using ELISA. Assessment of activity was performed using a standard protocol with 4-methylumbelliferyl-α-D-Biochem galactopyranoside, a blue pro-fluorogenic substrate (e.g., as described in Mapes and Sweeley, Biophys Res Commun. 53(4):1317-24 (1973); Hultberg et al., Acta Paediatr Scand. 64(1):123-31 (1975)).

Animals were euthanized 24 hours after intravenous injection. Following euthanasia (via CO₂ asphyxiation) and a terminal cardiac puncture blood collection; animals were perfused with PBS. Brain and liver were collected, rinsed briefly with PBS, and split/saved as follows.

1. Brain
a. One lobe was snap frozen in silanized polypropylene tubes (pre-weighed and containing 5 uL of inhibitor)
b. One lobe was sectioned so anatomical structures are preserved in both slices
   i. One section was preserved in formalin
   ii. One section was embedded in OCT medium

2. Liver
a. Left lobe was snap frozen in silanized polypropylene tubes (pre-weighed and containing 5 uL of inhibitor)
b. Right lobe was split with one half preserved in formalin and one half embedded in OCT medium (laid flat)
   Blood was collected via cardiac puncture and ˜500 uL and placed in a K2 EDTA anticoagulant tube (containing ˜10 uL ProBlock protease inhibitor) and inverted gently three times. Samples were centrifuged @ 3500 RPM, 2-8° C. for 15 minutes (process within 30 minutes of collection). Plasma was aliquoted at 120 uL into duplicate silanized polypropylene tubes at stored on dry ice and then moved to −70° C. until analysis.
   Statistical Analysis of Numerical in-Life Data was Assessed Using GraphPad Prism Software.

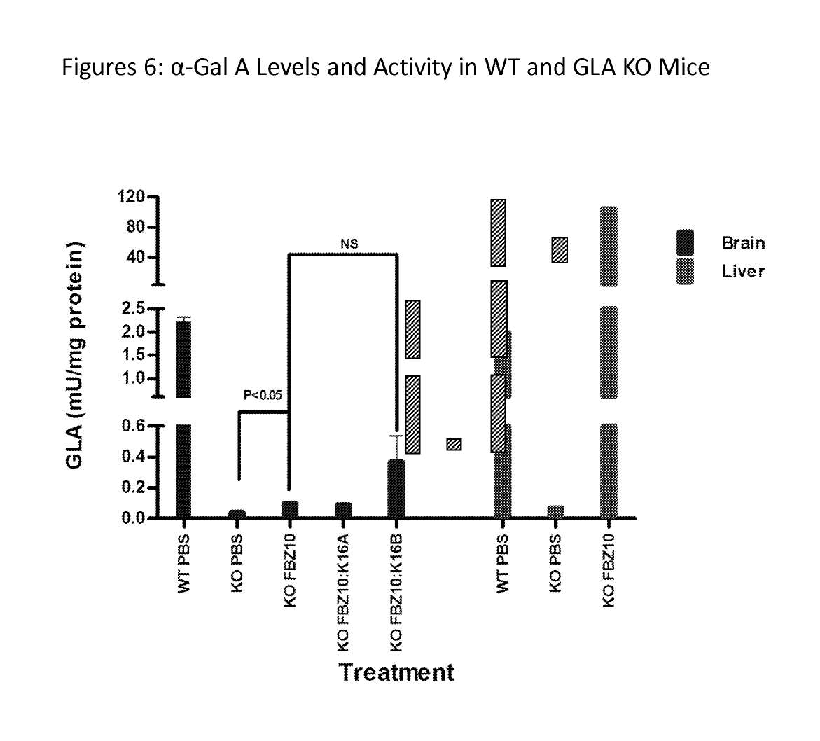

Assessment of α-Gal A activity in the brain showed dramatic reduction in activity in the GLA knockout animals as compared to the wild type animals, as expected. Injection of α-Gal A at 10 mg/kg led to an almost 3 fold increase in α-Gal A activity in the brain of the KO animals that was significant. Although addition of K16-ApoE peptide to the 10 mg/kg dose at a 1:2 molar ratio did not significantly affect brain penetration of α-Gal A in this study, technical issues may account for this result. However, as shown in FIG. 6 and Table 5B, an α-Gal A:K16-ApoE mixture at a 1:10 molar ratio (α-Gal A (10 mg/kg):K16-ApoE [1:10]) led to an increase in brain uptake as judged by α-Gal A activity. However, this change did not reach statistical significance due to variability in individual mouse response. The striped bars in FIG. 6 show α-Gal A activity in the liver, which were included as a control for injection as well as for α-Gal A activity.

| GLA KO | Brain GLA | Liver GLA | Liver GLA |
|---|---|---|---|
| Mouse | mU/mg | mU/mg | ng/mg protein |
| #1 | 0.07 | 158.53 | 1046.04 |
| #2 | 0.54 | 221.51 | 1122.23 |
| #3 | 0.76 | 263.99 | 1144.59 |
| #4 | 0.09 | 133.93 | 1014.19 |

### Example 6. Biodistribution of α-Gal A and K16Apo-E—High-Dose Study

Further experiments were conducted as described above with higher doses of α-Gal A, as shown in Table 6. The treatments were administered by intravenous injection in the tail vein of wild-type mice with slow bolus.

As in Example 5, the animals were euthanized 24 hours after injection. Blood was collected for serum by cardiac puncture, and the animals were perfused with PBS. The liver and brain were collected and frozen immediately. In group 3, two animals were lethargic and had difficulty recovering after the injection, and so were monitored for an hour; they were lethargic even up to six hours after the injection.

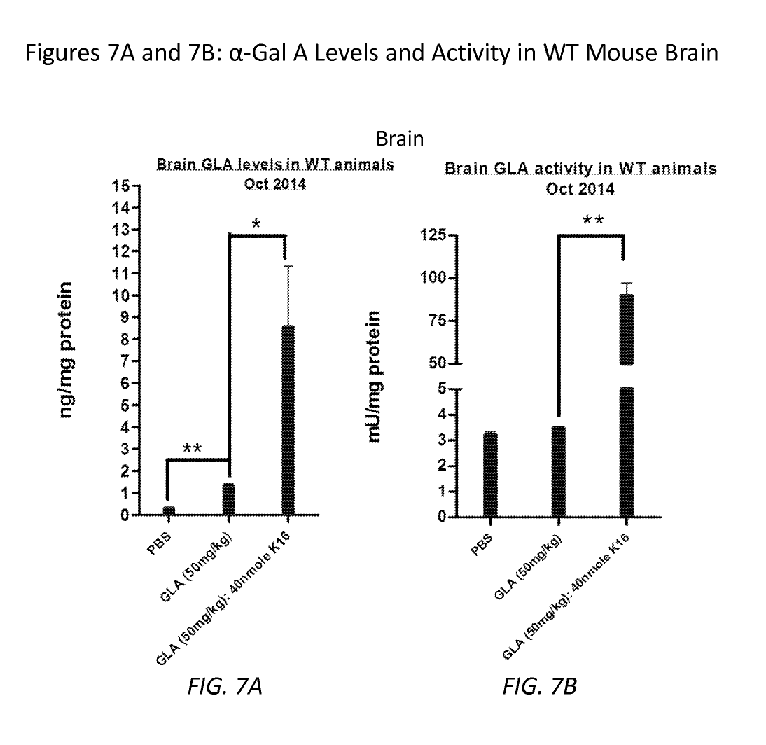

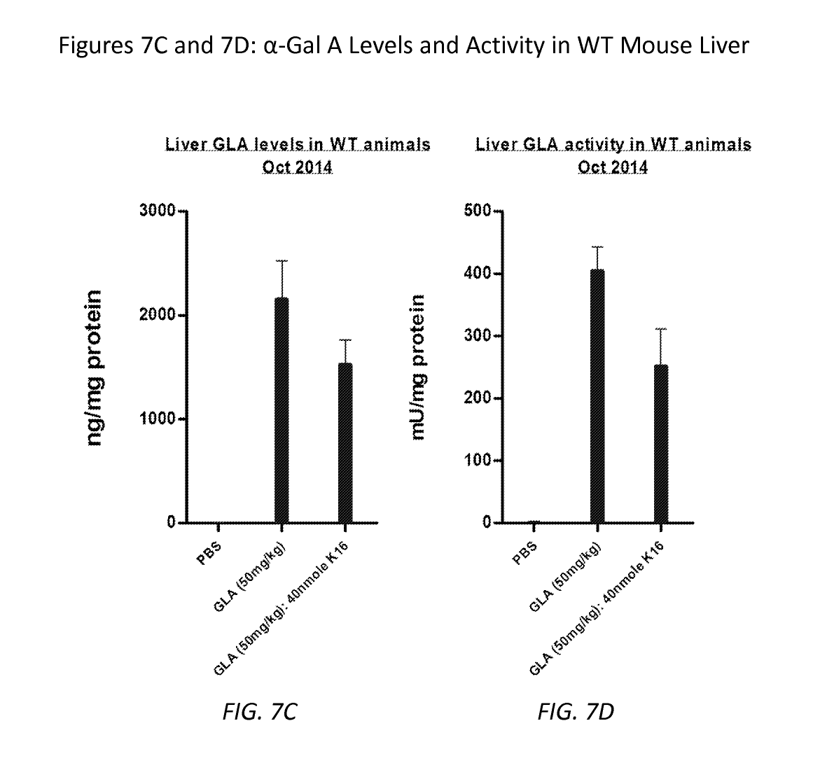

Assessment of α-Gal A levels by ELISA in WT animals showed an increase in α-Gal A levels in the brain when mice were injected with 50 mg/kg α-Gal A (FIG. 7A). A mixture of α-Gal A:K16-ApoE at a 1:3.6 molar ratio showed a very significant enhancement of α-Gal A enzyme brain penetration (FIG. 7B). The ability of K16-ApoE peptide to enhance α-Gal A brain penetration was also seen at the level of enzyme activity, assayed as described above. K16-ApoE led to more than 30 fold increase in α-Gal A enzyme activity in the brain when included in the mixture prior to injection (FIG. 7B). Liver GLA levels and activity did not differ significantly with the addition of K16-ApoE (FIGS. 7C-D), indicating that the increase in brain activity and levels with the peptide was not due to technical issues with the injection. If anything, α-Gal A levels and activity in the liver tended to be somewhat lower in mice treated with the α-Gal A:K16-ApoE mixture, possibly due to a shift in biodistribution of the injected enzyme.

| | | | | α-Gal A | K16Apo-E/ |
|---|---|---|---|---|---|
| Group | N | Mice | Description | Dose/mouse | mouse |
| 1 | 3 | C57B/L6 | saline control | 0 | 0 |
| 2 | 3 | C57B/L6 | α-Gal A alone | 1,150.00 ug | 0 |
| | | | (50 mg/kg) | | |
| 3 | 3 | C57B/L6 | α-Gal A | 1,150.00 ug | 181 ug |
| | | | (50 mg/kg | | |
| | | | [11.8 nmole]): | | |
| | | | 40 nmole | | |
| | | | K16-ApoE | | |

These results indicate that administration of α-Gal A formulated with K16-ApoE results in delivery of active α-Gal A to the mammalian brain.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of delivering a human protein to the central nervous system of a subject, the method comprising
administering a pharmaceutical composition comprising the human protein and a blood-brain barrier carrier peptide (BBB carrier peptide) to the subject, wherein the human protein is an enzymatically active human alpha-L-iduronidase (IDUA) protein, a human iduronate-2-sulfatase (IDS) protein, or a human α galactosidase A (α-Gal A) protein,
thereby delivering the human protein to the central nervous system of the subject.

2. A method of treating a subject having MPS I, Hunter syndrome, or Fabry disease, the method comprising
administering a pharmaceutical composition comprising the human protein and a blood-brain barrier carrier peptide (BBB carrier peptide) to the subject, wherein the human protein is an enzymatically active human alpha-L-iduronidase (IDUA) protein, a human iduronate-2-sulfatase (IDS) protein, or a human α galactosidase A (α-Gal A) protein,
thereby treating the subject having MPS I, Hunter syndrome, or Fabry disease.

3. The method of claim 1, wherein the BBB carrier peptide comprises a first portion comprising a transferrin-receptor binding site of a transferrin, or a receptor binding domain of an apolipoprotein, linked to a second portion comprising a hydrophilic segment of from 4-50 hydrophilic amino acids.

4. The method of claim 3, wherein the first portion comprises a receptor binding domain of an apolipoprotein, selected from the receptor binding domain of ApoA, ApoB, ApoC, ApoD, ApoE, ApoE2, ApoE3, and ApoE4.

5. The method of claim 3, wherein the hydrophilic amino acids are selected from the group consisting of arginine, asparagine, aspartic acid, glutamic acid, glutamine, histidine, lysine, serine, threonine, and tyrosine.

6. The method of claim 1, wherein the BBB carrier peptide comprises or consists of the sequence K-K-K-K-K-K-K-K-K-K-K-K-K-K-K-K-L-R-V-R-L-A-S-H-L-R-K-L-R-K-R-L-L-R-D-A (SEQ ID NO:45).

7. The method of claim 1,
wherein the IDUA protein comprises amino acids 20-653 of SEQ ID NO:53;
wherein the IDS protein comprises amino acids 26-550 of SEQ ID NO:55; or
wherein the α-galactosidase A protein comprises amino acids 32-429 of SEQ ID NO:56,

8. The method of claim 1, wherein the human protein and the BBB carrier peptide are present in a molar ratio of at least about 1:2, or higher.

9. The method of claim 1, wherein the human protein and the BBB carrier peptide are present in a molar ratio of about 1:10 to about 1:175.

10. The method of claim 1, wherein the human protein and the BBB carrier peptide are present in a molar ratio of about 1:155 to about 1:175.

11. The method of claim 10, wherein the human protein and the BBB carrier peptide are present in a molar ratio of about 1:167.

12. The method of claim 1, wherein
the IDUA protein is formulated for administration at a dose of about 0.2-50.0 mg of IDUA per kg of body weight;
the IDS protein is formulated for administration at a dose of about 0.2-50.0 mg of IDS per kg of body weight; or
the α-galactosidase A protein is formulated for administration at a dose of about 0.2-50.0 mg of α-galactosidase A per kg of body weight.

13. The method of claim 1, wherein the human protein is formulated for administration in an amount effective to reduce and/or arrest further accumulation of heparan sulfate levels in visceral tissue or urine of a subject.

14. The method of claim 1, wherein the human protein is an human α galactosidase A (α-Gal A) protein, and wherein the human α-galactosidase A protein is formulated for administration in an amount effective to reduce and/or arrest further accumulation of globotriaosylceramide and related glycosphingolipids in vascular endothelial lysosomes of a subject.

15. The method of claim 1, wherein the pharmaceutical composition comprises about 1.0 mg to about 65 mg of the human protein.

16. The method of claim 15, wherein the pharmaceutical composition comprises about 5 mg to about 60 mg of the human protein.

17. The method of claim 16, wherein the pharmaceutical composition comprises about 20 mg to about 45 mg of the human protein.

18. The method of claim 17, wherein the pharmaceutical composition comprises about 30 mg of the human protein.

19. The method of claim 1, wherein the pharmaceutical composition comprises about 10 mg to about 600 mg of the BBB carrier peptide.

20. The method of claim 19, wherein the pharmaceutical composition comprises about 75 mg to about 500 mg of the BBB carrier peptide.

21. The method of claim 20, wherein the pharmaceutical composition comprises about 375 mg of the BBB carrier peptide.

22. The method of claim 1, wherein the BBB carrier peptide is administered in a dose of about 5 mg/kg to about 8 mg/kg.

23. The method of claim 22, wherein the BBB carrier peptide is administered in a dose of about 6.5 mg/kg.

24. The method of claim 1, wherein the composition is administered intravenously, intramuscularly, or subcutaneously.

25. The method of claim 1, wherein the subject has Hurler Syndrome, Scheie Syndrome, or Hurler-Scheie Syndrome.

26. A method of producing a composition comprising a human protein and a blood-brain barrier carrier peptide (BBB carrier peptide), wherein the human protein is an enzymatically active human IDUA protein, IDS protein, or α galactosidase A protein, the method comprising:
culturing a host cell encoding an enzymatically active human protein under conditions permitting the production of the human protein,
recovering the human protein, and
combining the human protein with a blood-brain barrier carrier peptide.


SEQUENCE LISTING (56 sequences)

SEQ ID NO: 1 - protein, 4 aa, Artificial Sequence
  Description of Artificial Sequence Synthetic peptide
KKKK

SEQ ID NO: 2 - protein, 8 aa, Artificial Sequence
  Description of Artificial Sequence Synthetic peptide
KKKKKKKK

SEQ ID NO: 3 - protein, 12 aa, Artificial Sequence
  Description of Artificial Sequence Synthetic peptide
KKKKKKKKKKKK

SEQ ID NO: 4 - protein, 16 aa, Artificial Sequence
  Description of Artificial Sequence Synthetic peptide
KKKKKKKKKKKKKKKK

SEQ ID NO: 5 - protein, 4 aa, Artificial Sequence
  Description of Artificial Sequence Synthetic peptide
RRRR

SEQ ID NO: 6 - protein, 8 aa, Artificial Sequence
  Description of Artificial Sequence Synthetic peptide
RRRRRRRR

SEQ ID NO: 7 - protein, 12 aa, Artificial Sequence
  Description of Artificial Sequence Synthetic peptide
RRRRRRRRRRRR

SEQ ID NO: 8 - protein, 16 aa, Artificial Sequence
  Description of Artificial Sequence Synthetic peptide
RRRRRRRRRRRRRRRR

SEQ ID NO: 9 - protein, 4 aa, Artificial Sequence
  Description of Artificial Sequence Synthetic peptide
KRKR

SEQ ID NO: 10 - protein, 4 aa, Artificial Sequence
  Description of Artificial Sequence Synthetic peptide
KKKR

SEQ ID NO: 11 - protein, 12 aa, Artificial Sequence
  Description of Artificial Sequence Synthetic peptide
KKKRRRKKKRRR

SEQ ID NO: 12 - protein, 16 aa, Artificial Sequence
  Description of Artificial Sequence Synthetic peptide
KKKKRRRRKKKKRRRR

SEQ ID NO: 13 - protein, 20 aa, Artificial Sequence
  Description of Artificial Sequence Synthetic peptide
LRVRLASHLRKLRKRLLRDA

SEQ ID NO: 14 - protein, 23 aa, Artificial Sequence
  Description of Artificial Sequence Synthetic peptide
SSVIDALQYKLEGTTRLTRKRGL

SEQ ID NO: 15 - protein, 17 aa, Artificial Sequence
  Description of Artificial Sequence Synthetic peptide
KLATALSLSNKFVEGSH

SEQ ID NO: 16 - protein, 23 aa, Artificial Sequence
  Description of Artificial Sequence Synthetic peptide
YPAKPEAPGEDASPEELSRYYAS

SEQ ID NO: 17 - protein, 13 aa, Artificial Sequence
  Description of Artificial Sequence Synthetic peptide
LRHYLNLVTRQRY

SEQ ID NO: 18 - protein, 23 aa, Artificial Sequence
  Description of Artificial Sequence Synthetic peptide
AKPEAPGEDASPEELSRYYASLR

SEQ ID NO: 19 - protein, 11 aa, Artificial Sequence
  Description of Artificial Sequence Synthetic peptide
HYLNLVTRQRY

SEQ ID NO: 20 - protein, 23 aa, Artificial Sequence
  Description of Artificial Sequence Synthetic peptide
YPSDPDNPGEDAPAEDLARYYSA

SEQ ID NO: 21 - protein, 13 aa, Artificial Sequence
  Description of Artificial Sequence Synthetic peptide
LRHYINLITRQRY

SEQ ID NO: 22 - protein, 23 aa, Artificial Sequence
  Description of Artificial Sequence Synthetic peptide
APLEPVYPGDDATPEQMAQYAAE

SEQ ID NO: 23 - protein, 13 aa, Artificial Sequence
  Description of Artificial Sequence Synthetic peptide
LRRYINMLTRPRY

SEQ ID NO: 24 - protein, 20 aa, Artificial Sequence
  Description of Artificial Sequence Synthetic peptide
LRSRLASHLRKLRKRLLRDA

SEQ ID NO: 25 - protein, 20 aa, Artificial Sequence
  Description of Artificial Sequence Synthetic peptide
LRVRMASHLRKLRKRLLRDA

SEQ ID NO: 26 - protein, 20 aa, Artificial Sequence
  Description of Artificial Sequence Synthetic peptide
LRVRLATHLRKLRKRLLRDA

SEQ ID NO: 27 - protein, 20 aa, Artificial Sequence
  Description of Artificial Sequence Synthetic peptide
LRVRLASHLRKLPKRLLRDA

SEQ ID NO: 28 - protein, 20 aa, Artificial Sequence
  Description of Artificial Sequence Synthetic peptide
LRVRLASHLRKLRKRLMRDA

SEQ ID NO: 29 - protein, 20 aa, Artificial Sequence
  Description of Artificial Sequence Synthetic peptide
LRVRLASHLRNLRKRLLRDA

SEQ ID NO: 30 - protein, 20 aa, Artificial Sequence
  Description of Artificial Sequence Synthetic peptide
LRVRLASHLRKVRKRLLRDA

SEQ ID NO: 31 - protein, 20 aa, Artificial Sequence
  Description of Artificial Sequence Synthetic peptide
LRVRMSSHLRKLRKRLLRDA

SEQ ID NO: 32 - protein, 20 aa, Artificial Sequence
  Description of Artificial Sequence Synthetic peptide
LRVRLASHLRNVRKRLLRDA

SEQ ID NO: 33 - protein, 20 aa, Artificial Sequence
  Description of Artificial Sequence Synthetic peptide
LRVRLASHLRNMRKRLLRDA

SEQ ID NO: 34 - protein, 20 aa, Artificial Sequence
  Description of Artificial Sequence Synthetic peptide
LRARMASHLRKLRKRLLRDA

SEQ ID NO: 35 - protein, 20 aa, Artificial Sequence
  Description of Artificial Sequence Synthetic peptide
LRVRLSSHLRKLRKRLMRDA

SEQ ID NO: 36 - protein, 20 aa, Artificial Sequence
  Description of Artificial Sequence Synthetic peptide
LRSRLASHLRKLRKRLMRDA

SEQ ID NO: 37 - protein, 20 aa, Artificial Sequence
  Description of Artificial Sequence Synthetic peptide
LRVRLSSHLPKLRKRLLRDA

SEQ ID NO: 38 - protein, 20 aa, Artificial Sequence
  Description of Artificial Sequence Synthetic peptide
LRVRLASHLRKMRKRLMRDA

SEQ ID NO: 39 - protein, 20 aa, Artificial Sequence
  Description of Artificial Sequence Synthetic peptide
LRVRLASHLRNLPKRLLRDA

SEQ ID NO: 40 - protein, 20 aa, Artificial Sequence
  Description of Artificial Sequence Synthetic peptide
LRLRLASHLRKLRKRLLRDL

SEQ ID NO: 41 - protein, 20 aa, Artificial Sequence
  Description of Artificial Sequence Synthetic peptide
LRVRLANHLRKLRKRLLRDL

SEQ ID NO: 42 - protein, 24 aa, Artificial Sequence
  Description of Artificial Sequence Synthetic peptide
KKKKLRVRLASHLRKLRKRLLRDA

SEQ ID NO: 43 - protein, 28 aa, Artificial Sequence
  Description of Artificial Sequence Synthetic peptide
KKKKKKKKLRVRLASHLRKLRKRLLRDA

SEQ ID NO: 44 - protein, 32 aa, Artificial Sequence
  Description of Artificial Sequence Synthetic polypeptide
KKKKKKKKKKKKLRVRLASHLRKLRKRLLRDA

SEQ ID NO: 45 - protein, 36 aa, Artificial Sequence
  Description of Artificial Sequence Synthetic polypeptide
KKKKKKKKKKKKKKKKLRVRLASHLRKLRKRLLRDA

SEQ ID NO: 46 - protein, 40 aa, Artificial Sequence
  Description of Artificial Sequence Synthetic polypeptide
KKKKKKKKKKKKKKKKKKKKLRVRLASHLRKLRKRLLRDA

SEQ ID NO: 47 - protein, 44 aa, Artificial Sequence
  Description of Artificial Sequence Synthetic polypeptide
KKKKSSVIDALQYKLEGTTRLTRKRGLKLATALSLSNKFVEGSH

SEQ ID NO: 48 - protein, 48 aa, Artificial Sequence
  Description of Artificial Sequence Synthetic polypeptide
KKKKKKKKSSVIDALQYKLEGTTRLTRKRGLKLATALSLSNKFVEGSH

SEQ ID NO: 49 - protein, 52 aa, Artificial Sequence
  Description of Artificial Sequence Synthetic polypeptide
KKKKKKKKKKKKSSVIDALQYKLEGTTRLTRKRGLKLATALSLSNKFVEGSH

SEQ ID NO: 50 - protein, 56 aa, Artificial Sequence
  Description of Artificial Sequence Synthetic polypeptide
KKKKKKKKKKKKKKKKSSVIDALQYKLEGTTRLTRKRGLKLATALSLSNKFVEGSH

SEQ ID NO: 51 - protein, 60 aa, Artificial Sequence
  Description of Artificial Sequence Synthetic polypeptide
KKKKKKKKKKKKKKKKKKKKSSVIDALQYKLEGTTRLTRKRGLKLATALSLSNKFVEGSH

SEQ ID NO: 52 - protein, 20 aa, Artificial Sequence
  Description of Artificial Sequence Synthetic peptide
LRKLRKRLLRLRKLRKRLLR

SEQ ID NO: 53 - protein, 653 aa, Homo sapiens
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 54 - DNA, 1962 bp, Homo sapiens
atgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatga

SEQ ID NO: 55 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 56 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL